(19) United States
(12) Patent Application Publication
(10) Pub. No.: US 2012/0141538 A1
(43) Pub. Date: Jun. 7, 2012

(54) MODULATORS OF THE CX3CRI RECEPTOR AND THERAPEUTIC USES THEREOF

(75) Inventors: Guy Gorochov, Paris (FR); Karim Dorgham, Ivry Sur Seine (FR); Christophe Combadiere, Magny Le Hongre (FR); Philippe Deterre, Paris (FR)

(73) Assignee: UNIVERSITE PIERRE ET MARIE CURIE -PARIS VI, Paris (FR)

(21) Appl. No.: 13/140,615

(22) PCT Filed: Dec. 16, 2009
(86) PCT No.: PCT/EP09/67325

§ 371 Date: Feb. 22, 2012

(30) Foreign Application Priority Data
Dec. 17, 2008 (IB) PCT/IB2008/055683

(51) Int. Cl. A61K 38/08; C07K 14/00; C07K 16/46; C07H 21/04; A61K 31/7088; A61K 39/39; A61P 25/28; A61P 25/00; A61P 31/12; A61P 35/00; A61P 29/00; A61P 37/00; A61P 9/00; A61P 37/06; A61P 19/02; A61P 1/04; A61P 9/10; A61P 31/18; A61P 17/02; C07K 7/06

(52) U.S. Cl. 424/278.1; 530/329; 530/324; 530/387.3; 536/23.1; 514/21.7; 514/44 R; 514/17.7; 514/17.5; 514/3.7; 514/19.3; 514/18.3; 514/17.9; 514/16.6; 514/13.2; 514/1.9; 514/19.4; 514/3.8; 514/19.2; 514/18.6; 435/69.1

(57) ABSTRACT

The present invention concerns modulators of the CX3CR1 receptor. More specifically, antagonists and agonists of the CX3CR1 receptor have been identified. These antagonists and agonists can be used for treating an inflammatory disorder, an autoimmune disorder, a cardiovascular disease, a neurodegenerative disease, a graft versus host disease, a behavioral disorder, a cicatrisation disorder, a viral infection, cancer or pain. They may also be used as an adjuvant in a vaccine composition.

### FIELD OF THE INVENTION

The present invention concerns modulators of the CX3CR1 receptor. More specifically, antagonists and agonists of the CX3CR1 receptor have been identified. These antagonists and agonists can be used for treating an inflammatory disorder, an autoimmune disorder, a cardiovascular disease, a neurodegenerative disease, a graft versus host disease, a behavioral disorder, a cicatrisation disorder, a viral infection, cancer or pain. They may also be used as an adjuvant in a vaccine composition.

### BACKGROUND OF THE INVENTION

Chemokines are a family of small secreted proteins (typically 8-10 kDa) that are involved in leukocyte trafficking in homeostatic and inflammatory conditions. They, together with their receptors, have been identified as targets for modulating leukocyte migration in physiological or pathological conditions.

The chemokine CX3CL1 (also referred to as fractalkine) is structurally distinctive from other chemokines in that it exists both as soluble and as membrane-anchored forms (Imai et al. (1997) Cell 91, 521-530). Membrane-anchored, it promotes strong selectin- and integrin-independent adhesion of leukocytes that express CX3CR1, its sole receptor. Soluble CX3CL1, on the other hand, which is produced by the cleavage of native CX3CL1, is a potent chemoattractant.

Proteins such as vMIP-II encoded by the Kaposi's sarcoma-associated herpesvirus (Kledal et al. 1997, Science 277(5332): 1656-9; Chen et al. 1998, J Exp Med 188(1): 193-8) and the RSV G protein (Harcourt et al. 2006, J Immunol 176(3): 1600-8) have been shown to bind to CX3CR1 and/or CX3CL1 and to modulate their activity. However, their binding affinity to CX3CR1 and/or CX3CL1 is low and they do therefore not constitute suitable candidate therapeutic compounds.

Mutated CX3CL1 proteins have been described by Mizoue et al. (2001, J Biol Chem 276, 33906-33914), Davis et al. (2004, Mol. Pharmacol. 66, 1431-1439) and by Inoue et al. (2005, Arthritis Rheum 52, 1522-33). More specifically, Mizoue et al. (2001, J Biol Chem 276, 33906-33914) reported N-terminal modifications to CX3CL1 that result in proteins with reduced biological activity. Davis et al. (2004) described both a chimeric CX3CL1-MIP-II fusion protein and a mutated human CX3CL1 protein in which amino acids 9-11 had been deleted. However, these proteins exhibited poor apparent binding affinities. Inoue et al. (2005) reported that a murine CX3CL1 lacking the four N-terminal residues behaves as an antagonist for murine CX3CR1. However, there is no indication that this compound also binds human CX3CR1. Moreover removal of the first seven residues of human CX3CL1 has been shown to produce an inactive analogue with poor affinity for CX3CR1 (Mizoue et al., 2001).

CX3CR1 and CX3CL1 have been implicated in a number of inflammatory diseases (Umehara et al. 2001, Trends Immunol 22, 602-7; Stievano et al. 2004, Crit. Rev Immunol 24, 205-28). For example, in endothelial cells, both stress and inflammatory cytokines up-regulate CX3CL1 expression (Umehara et al. 2004, Arterioscler Thromb Vasc Biol 24, 34-40). The recruitment of CX3CR1-expressing leukocytes such as cytotoxic CD8 T cells and NK cells to glomeruli appears to be associated with both glomerulonephritis (Chen et al. 1998, J. Exp. Med. 188, 193-198) and lupus nephritis (Inoue et al. 2005, Arthritis Rheum 52, 1522-33). Moreover, the inflamed vascular CX3CL1+-endothelium captures CX3CR1+-monocytes, which become a major component of the cell accumulation that leads to atherogenesis both in mice (Combadiere et al. 2003, Circulation 107, 1009-16; Lesnik et al. 2003, J Clin Invest 111, 333-40) and in humans (Moatti et al. 2001, Blood 97, 1925-8). Finally, CX3CR1+-leukocyte recruitment plays a role in both rheumatoid arthritis (Ruth et al. 2001, Arthritis Rheum 44, 1568-81) and inflammatory bowel disease (Brand et al. 2006, Am J Gastroenterol 101, 99-106).

Hence CX3CR1 modulators are promising anti-inflammatory drugs, and there is a need in the art for identifying novel and potent CX3CR1 modulators.

### DESCRIPTION OF THE INVENTION

A phage display strategy has been used to identify both agonistic and antagonistic CX3CR1 modulators. Several agonistic and antagonistic CX3CR1 modulators have been identified (SEQ ID Nos. 7-50). These CX3CR1 modulators allowed defining consensus sequences for CX3CR1 modulators (SEQ ID Nos. 1-6 and 51). The CX3CR1 modulators described herein exhibit an apparent CX3CR1 binding affinity that is close to that of native human CX3CL1. Moreover, these CX3CR1 modulators are fully recombinant CX3CL1 analogs, which only contain naturally-occurring amino acids, and are thus amenable with low cost production.

One antagonistic modulator so identified (referred to as F1) was further characterized. F1 specifically bound to human cells expressing CX3CR1 and had a Kd value close to that of native CX3CL1. However, F1 is not a signaling molecule since it did not induce chemotaxis, calcium flux or CX3CR1 internalization. Moreover, it potently inhibited the CX3CL1-induced calcium flux and chemotaxis in CX3CR1-expressing primary cells of both human and murine origin, with an IC50 of 5-50 nM. It also efficiently inhibited the cell adhesion mediated by the CX3CL1-CX3CR1 axis. Finally, Fl partially inhibited peritoneal recruitment of CX3CR1+ monocytes in a non-infectious murine model of peritonitis.

The present invention thus relates to agonists and antagonists of human CX3CR1. Such modulators can be used as lead compounds for the development of anti-inflammatory drugs that act by inhibiting CX3CR1.

Modulators in Accordance with the Invention

The present invention relates to an isolated and/or purified modulator of a human CX3CR1 receptor comprising a sequence X₁-X₂-X₃-X₄-X₅-X₆-X₇ (SEQ ID NO: 1) or X₁-X₃-X₄-X₅-X₆-X₇ (SEQ ID NO:2) wherein:
—X₁ is I, T, F, Q, S, W, A, G, N or V;
X₂, when present, is L, P or R;
X₃ is D, A, Q, G, L, I, P, H, F, V or S;
X₄ is N, Q, G, S, L, R, F, H, V, M, Y or P;
X₅ is V, A, D or G;
X₆ is L, M or V; and
X₇ is S, P, T or A;
   wherein SEQ ID NO: 2 does not consist of the sequence QHHGVT (SEQ ID NO: 52) or QHLGMT (SEQ ID NO: 53). Said sequence of SEQ ID NO: 1 or SEQ ID NO:2 is preferably located at the N-terminal extremity of said modulator.

As used herein, the term “modulator” refers to a compound that binds to the CX3CR1 receptor and that modulates its biological activity. The modulator may either corresponds to antagonist (i.e. it reduces or inhibits the biological activity of the CX3CR1 receptor) or to an agonist (i.e. it induces or increases the biological activity of the CX3CR1 receptor).

As used herein, the term “CX3CR1 receptor” refers to the receptor of the CX3CL1 chemokine. The CX3CR1 receptor is encoded by the CX3CR1 gene, which is located at human chromosome location 3p21.3 (Entrez GenelD: 1524). The term “CX3CR1 receptor” refers to the protein of SEQ ID NO: 63 and to naturally-occurring variants thereof such a e.g. splice variants, polymorphic variants and variants obtained through proteolytic processing. Such naturally-occurring variants are shown in e.g. SwissProt Accession No. P49238.

As used herein, the term “biological activity” of the CX3CR1 receptor refers to any of the biological activities mediated by the activation of the CX3CR1 receptor by the CX3CL1 chemokine such as, e.g., mobilization of intracellular calcium and/or induction of chemotaxis of CD8+ T cells and/or NK cells. Methods for measuring the biological activity of the CX3CR1 receptor are well known in the art. For example, a calcium mobilization assay, a chemotaxis assay or an in vivo thioglycollate-induced inflammation assay may be used to measure the biological activity of the CX3CR1 receptor. Such assays are described in Example 1.

The modulators according to the invention bind to the CX3CR1 receptor. Preferably, they specifically bind to the CX3CR1 receptor. Most preferably, their apparent binding affinity (IC₅₀) is of less than 10, 5, 2.5, 2.3, 2, 1.9, 1.5, 1, 0.5, 0.3, 0.16 or 0.1 nM. The apparent binding affinity is preferably measured by comparison to that of native CX3CL1 in a competition binding, with HEK-CX3CR1 or CHO-CX3CR1 cells and [¹²⁵I]-CX3CL1 as a tracer.

Antagonists are not internalized into CX3CR1-expressing cells and do therefore not transmit any signal. On the contrary, agonists are internalized into CX3CR1-expressing cells and do therefore transmit a signal.

More specifically, an “antagonist” according to the invention is capable of (i) inhibiting CX3CL1-induced calcium response in PBMC cells, (ii) inhibiting CX3CL1-induced chemotaxis of NK cells and of CD8+ T cells, and/or (iii) decreasing monocyte (e.g. CD11b+Ly6G-7/4+ monocytes, most preferably 7/4^{lo} monocytes) recruitment, preferably in a dose-dependant manner. The presence of an antagonist preferably reduces the biological activity of the CX3CR1 receptor by at least 10, 15, 20, 25, 30, 40 or 50% as compared to the biological activity of the CX3CR1 receptor in the presence of CX3CL1 only.

An “agonist” according to the invention is capable of (i) inducing a calcium response in cells, for example in PBMC cells, (ii) inducing chemotaxis of NK cells and of CD8+ T cells, and/or (iii) inducing monocyte (e.g. CD11b+Ly6G-7/4+ monocytes, most preferably 7/4^{lo} monocytes) recruitment, preferably in a dose-dependant manner. The agonist preferably enhances the biological activity of the CX3CR1 receptor by at least 10, 15, 20, 25, 30, 40 or 50% as compared to the biological activity of the CX3CR1 receptor in the presence of CX3CL1.

Unless otherwise indicated, the term “N-terminal extremity” refers to the extremity of the mature isoform of a polypeptide.

The term “mature isoform of a polypeptide” refers to the isoform of a polypeptide generated after cleavage of the signal peptide, propeptide or pre-propeptide.

A polypeptide comprising a given sequence at its N-terminal extremity refers to a polypeptide in which the first amino acids of the mature isoform consist in said sequence. For example, a polypeptide comprising the sequence ILDNGVS (SEQ ID NO: 8) at its N-terminal extremity refers to a polypeptide in which the seven first residues of the mature isoform of the polypeptide are ILDNGVS. In other terms, the most N-terminal (first) amino acid of the mature isoform of such a polypeptide is an isoleucine.

Unless otherwise indicated (e.g. by reference to a sequence of the sequence listing), the position of an amino acid within a polypeptide is given relatively to the mature isoform of said polypeptide.

As used herein, “isolated and/or purified” refers to a compound that is isolated and/or purified from the human body and/or from a library of compounds.

In a preferred embodiment, the modulator according to the invention is an antagonist. Such an antagonist is preferably selected from the group consisting of any one of (i) to (vi):
(i) an antagonist comprising a sequence X₁-X₂-X₃-X₄-X₅-X₆-X₇ (SEQ ID NO: 1) or X₁-X₃-X₄-X₅-X₆-X₇ (SEQ ID NO: 2) wherein:
   X₁ is I, T, F, Q, S, W or V;
   X₂, when present, is L, P or R;
   X₃ is D, A, Q, G, L, I, P or S;
   X₄ is N, Q, G, S, L, R, F, H or P;
   X₅ is V, A, D or G;
   X₆ is L or V; and
   X₇ is S, P, T or A;
(ii) an antagonist comprising a sequence X₁-X₂-X₃-X₄-X₅-X₆-X₇ (SEQ ID NO: 1) wherein:
   X₁ is I, T, F, Q, S or W;
   X₂ is L, P or R;
   X₃ is D, A, Q, G, L, I, P or S;
   X₄ is N, Q, G, S, L, R, F, H or P;
   X₅ is V, A, D or G;
   X₆ is L or V; and
   X₇ is S, P, T or A;
(iii) an antagonist comprising a sequence X₁-X₃-X₄-X₅-L-X₇ (SEQ ID NO: 3) wherein:
   X₁ is Q or V;
   X₃ is Q, L or S;
   X₄ is S, L, or F;
   X₅ is V or A; and
   X₇ is S or P;
(iv) an antagonist comprising a sequence X₁-L-X₃-X₄-X₅-X₆-X₇ (SEQ ID NO: 4) wherein:
   X₁ is I, T, F, S or W;
   X₃ is D, A, Q, G, I, P or S;
   X₄ is N, Q, G, S, L, R, H or P;
   X₅ is V, D or G;
   X₆ is L or V; and
   X₇ is S, P, T or A;
(v) an antagonist comprising a sequence Q-X₂-X₃-X₄-X₅-X₆-A (SEQ ID NO: 5) wherein:
   X₂ is P or R;
   X₃ is D or L;
   X₄ is S or F;
   X₅ is V or A; and
   X₆ is L or V;
(vi) an antagonist comprising a sequence I-L-D-X₄-G-X₆-X₇ (SEQ ID NO: 6) wherein:
   X₄ is any amino acid;
   X₆ is L or V; and
   X₇ is A or S.

As immediately apparent to the skilled in the art, the sequences of SEQ ID Nos. 1 to 6 defined in (i) to (vi) hereabove correspond to specific embodiments of the sequences of general formula X₁-X₂-X₃-X₄-X₅-X₆-X₇ (SEQ ID NO: 1) or X₁-X₃X₄-X₅-X₆-X₇ (SEQ ID NO: 2) wherein:
X₁ is I, T, F, Q, S, W, A, G, N or V;
X₂, when present, is L, P or R;
X₃ is D, A, Q, G, L, I, P, H, F, V or S;
X₄ is N, Q, G, S, L, R, F, H, V, M, Y or P;
X₅ is V, A, D or G;
X₆ is L, M or V; and
X₇ is S, P, T or A;
   and wherein SEQ ID NO: 2 does not consist of the sequence QHHGVT (SEQ ID NO: 52) or QHLGMT (SEQ ID NO: 53). In other terms, the sequences of general formula X₁-X₂-X₃-X₄-X₅-X₆-X₇ (SEQ ID NO: 1) or X₁-X₃-X₄-X₅-X₆-X₇ (SEQ ID NO: 2) are preferably selected from the sequences as defined in (i) to (vi) hereabove. Said sequences defined in (i) to (vi) hereabove are thus preferably located at the N-terminal extremity of the modulator according to the invention.

Most preferably, said antagonist comprises a sequence selected from the group consisting of SEQ ID Nos. 8-24. In this preferred embodiment, the sequences of general formula X₁-X₂-X₃-X₄-X₅-X₆-X₇ (SEQ ID NO: 1) or X₁-X₃-X₄-X₅-X₆-X₇ (SEQ ID NO: 2) are selected from the sequences of SEQ ID Nos. 8-24.

In another preferred embodiment, the modulator according to the invention is an agonist. Such an agonist preferably comprises a sequence X₁-X₃-X₄-X₅-X₆-X₇ (SEQ ID NO:2) in which:
X₁ is Q, A, G or N;
X₃ is P, A, H, L, S, F or V;
X₄ is G, Q, V, M, L, S, P, H, R or Y;
X₅ is A or G;
X₆ is L, M or V; and
X₇ is S, P, T or A.

As immediately apparent to the skilled in the art, the sequence of SEQ ID NO: 2 hereabove also corresponds to a specific embodiment of the sequences of general formula X₁-X₂-X₃-X₄-X₅-X₆-X₇ (SEQ ID NO: 1) or X₁-X₃-X₄-X₅-X₆-X₇ (SEQ ID NO: 2). It is thus preferably located at the at the N-terminal extremity of the modulator according to the invention.

Most preferably, said agonist comprises a sequence selected from the group consisting of SEQ ID Nos. 25-51. In this preferred embodiment, the sequences of general formula X₁-X₂-X₃-X₄-X₅-X₆-X₇ (SEQ ID NO: 1) or X₁-X₃-X₄-X₅-X₆-X₇ (SEQ ID NO: 2) are selected from the sequences of SEQ ID Nos. 25-51.

Alternatively, the agonist according to the invention may comprise a sequence X₁-X₃-X₄-X₅-X₆-X₇ (SEQ ID NO: 7) in which:
X₁ is any amino acid;
X₃ is any amino acid;
X₄ is any amino acid;
X₅ is V, A, D or G;
X₆ is L, M or V; and
X₇ is S, P, T or A;
   wherein SEQ ID NO: 7 does not consist of the sequence QHHGVT (SEQ ID NO: 52) or QHLGMT (SEQ ID NO: 53). Said sequence of SEQ ID NO: 7 is preferably located at the N-terminal extremity of said modulator.

The modulator according to the invention may correspond either to a peptide or to a polypeptide.

In a preferred embodiment, said modulator corresponds to a peptide (i.e. a chain of amino acids of less than 50, 40, 30, 20 or 10 amino acids). The peptide of the invention may optionally comprise chemical modifications improving its stability and/or its biodisponibility. Such chemical modifications aim at obtaining peptides with increased protection of the peptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to modifications to the N-terminal and/or C-terminal ends of the peptides, modifications at the amide bond between two amino acids, modifications at the alpha carbon of the amide bond linking two amino acids, chirality changes, retro-inversions, modifications yielding azapeptides and modifications yielding betapeptides.

In another preferred embodiment, said modulator corresponds to a polypeptide (i.e. a chain of amino acids of more than 50 amino acids). Said polypeptide preferably corresponds to a soluble polypeptide.

The modulator according to the invention preferably consists of a peptide or a polypeptide comprising a fragment of at least 10, 20, 30, 40, 50, 60, 70, 80, 90, 100, 125, 150, 175, 200, 250 or 300 amino acids of SEQ ID NO: 55 or 56. The modulator according to the invention preferably comprises the chemokine domain of CXC3CL1. The modulator may further comprise the mucin-like stalk domain of CXC3CL1. Thus preferred modulators comprise or consist of:
i. amino acids 1 to 77, 1 to 316 or 1 to 318 of SEQ ID NO: 55;
ii. amino acids 1 to 76, 1 to 315 or 1 to 317 of SEQ ID NO: 56;
iii. a sequence exhibiting at least 80, 85, 90, 95, 96, 97, 98 or 99% identity to (i) or (ii).

The information obtained from the examples presented herein can be used for the construction of second-generation libraries in which:
the N-terminal extremity of the modulator is identical to the N-terminal extremity of SEQ ID NO: 55 or 56; and
additional mutations are introduced into the region proximal to the C-terminal to the first pair of conserved cysteines (the N-loop region). This region approximately corresponds to residues 40-50 of CX3CL1. However, additional mutations are preferably neither introduced in residues that are clearly located within the core of the protein, nor in the four conserved cysteine residues.

The modulator according to the invention preferably has an N-terminal extremity consisting of the sequence of any one of SEQ ID NOs. 7 to 51. The modulator according to the invention may also have an N-terminal extremity consisting of the sequence of any one of SEQ ID NOs. 1 to 6 as defined hereabove.

The modulator according to the invention may further comprise a fragment of an immunoglobulin. Such fragments of an immunoglobulin are useful either for enhancing solubility or for targeting the modulator to a specific organ (see e.g. Challita-Eid et al. 1998, J Immunol 161(7): 3729-36; Biragyn et al. 1999, Nat Biotechnol 17(3): 253-8.).

The modulator may further comprise a leader sequence such as e.g. a signal peptide, a propeptide or a pre-propeptide, wherein said leader sequence is cleaved off upon proteolytic processing, thereby generating a peptide or a polypeptide having an N-terminal extremity according to the invention.

Mutants in Accordance with the Invention

The invention further relates to an isolated and/or purified mutant of a human

CX3CL1 polypeptide characterized in that the N-terminal extremity of a mature isoform of said mutant:
consists of the sequence of any one of SEQ ID NOs. 1 to 51; and
does not consist of QHHGVT (SEQ ID NO: 52) or QHLGMT (SEQ ID NO: 53).

As used herein, the terms “CX3CL1” and “CX3CL1 polypeptide” refer to the human CX3CL1 chemokine. The CX3CL1 chemokine is encoded by the CX3CL1 gene, which is located at human chromosome location 16q13 (Entrez GeneID: 6376). More specifically, “CX3CL1” and “CX3CL1 polypeptide” refer to the protein of SEQ ID NO: 54 and to naturally-occurring variants thereof such a e.g. splice variants, polymorphic variants and variants obtained through proteolytic processing. Such naturally-occurring variants are shown in e.g. SwissProt Accession No. P78423.

As used herein, the term “mutant” refers to a non naturally-occurring variant of a polypeptide.

The mutants in accordance with the invention are characterized by their N-terminal extremity, which consist of the sequence of any one of SEQ ID NOs. 1 to 51.

The mutants in accordance with the invention may correspond to fragments of the CX3CL1 polypeptide. The mutants may for example comprise or consist of a fragment of at least 10, 20, 30, 40, 50, 60, 70, 80, 90, 100, 125, 150, 175, 200, 250 or 300 amino acids of a CX3CL1 polypeptide. The mutants preferably correspond to a soluble fragment of a CX3CL1 polypeptide. For example, the mutants may comprise the chemokine domain and/or the mucin-like stalk domain of CXC3CL1. Thus preferred mutants comprise amino acids 31 to 100, 31 to 339 or 31 to 341 of a CX3CL1 polypeptide of SEQ ID NO: 54.

Other preferred mutants comprise a sequence exhibiting at least 80, 85, 90, 95, 96, 97, 98 or 99% identity to a fragment of at least 10, 20, 30, 40, 50, 60, 70, 80, 90, 100, 125, 150, 175, 200, 250 or 300 amino acids of a CX3CL1 polypeptide, for example to amino acids 31 to 100, 31 to 339, 31 to 341 or 31 to 397 of SEQ ID NO: 54.

Mutants consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the mutant consisting of an amino acid sequence at least 80%, 85%, 90%, 95%, 96%, 97%, 98% or 99% identical to a reference sequence may correspond to a homologous sequence derived from another mammalian species than the reference sequence. In another preferred embodiment, the substitution preferably corresponds to a conservative substitution as indicated in the table below.

| Conservative substitutions | Type of Amino Acid |
|---|---|
| Ala, Val, Leu, Ile, Met, Pro, | Amino acids with aliphatic hydrophobic |
| Phe, Trp | side chains |
| Ser, Tyr, Asn, Gln, Cys | Amino acids with uncharged but polar |
| | side chains |
| Asp, Glu | Amino acids with acidic side chains |
| Lys, Arg, His | Amino acids with basic side chains |
| Gly | Neutral side chain |

The mutants may comprise a fragment of an immunoglobulin and/or a leader sequence. The leader sequence may either correspond to the native CX3CL1 signal peptide or to a heterologous sequence.

Most preferred mutants correspond to modulators according to the invention. More specifically, such mutants can bind to the CX3CR1 receptor and modulate its biological activity as described in the paragraph entitled “Modulators in accordance with the invention”.

Nucleic Acids in Accordance with the Invention

The invention is further directed to a nucleic acid encoding the modulator or the mutant according to the invention. Such nucleic acids can readily be obtained by the skilled in the art by cloning and directed mutagenesis of SEQ ID NO: 58.

Therapeutic Use of Modulators, Mutants and Nucleic Acids in Accordance with the Invention

CX3CL1 and its receptor CX3CR1 play a major role in numerous inflammatory processes. The CX3CL1/CX3CR1 pathway has been shown to be involved in the development of autoimmune diseases such as multiple sclerosis (Huang et al. 2006, Faseb J 20(7): 896-905), rheumatoid arthritis (Sawai et al. 2007 Arthritis Rheum 56(10): 3215-25), lupus erythematosus (Inoue et al. 2005, Arthritis Rheum 52(5): 1522-33), cardiovascular diseases (Moatti et al. 2001, Blood 97(7): 1925-8; Combadiere et al. 2003, Circulation 107(7): 1009-16; Lesnik et al. 2003, J Clin Invest 111(3): 333-40; McDermott et al. 2003, J Clin Invest 111(8): 1241-50), neurodegenerative diseases such as macular degeneration (Combadiere et al. 2007, J Clin Invest 117(10): 2920-8) and Parkinson's disease (Cardona et al. 2006, Nat Neurosci 9(7): 917-24), graft versus host disease (Robinson et al. 2000, J Immunol 165(11): 6067-72), cancer (Andre et al. 2006, Ann Oncol 17(6): 945-51; Vitale et al. 2007, Gut 56(3): 365-72), viral infections such as HIV infections (Faure et al. 2000, Science 287(5461): 2274-7; Garin et al. 2003, J Immunol 171(10): 5305-12), respiratory syncytial virus infections (Tripp et al. 2001, Nat Immunol 2(8): 732-8) and West Nile Virus infections (Getts et al. 2008, J Exp Med 205(10): 2319-37). The CX3CL1/CX3CR1 pathway has also been shown to be involved in cicatrisation (Ishida et al. 2008, J Immunol 180(1): 569-79), pain (Holmes et al. 2008, J Neurochem 106(2): 640-9), behavioral disorders (Gordon Research Conferences, 21-26 Sep. 2008), and nucleic acids encoding CXC3CL1 are useful as adjuvant in vaccines (Iga et al. 2007, Vaccine 25(23): 4554-63).

Therefore, the invention is directed to a modulator, a mutant or a nucleic acid according to the invention for use as a medicament, more specifically for use for the treatment or the prevention of any disease described herein.

A preferred aspect of the invention is directed to:
a method for treating or preventing a disease selected from the group consisting of an inflammatory disorder, an autoimmune disorder, a cardiovascular disease, a neurodegenerative disease, a graft versus host disease, a behavioral disorder, a cicatrisation disorder, a viral infection, cancer and pain comprising the step of administering an effective amount of a modulator, a mutant or a nucleic acid according to the invention to an individual in need thereof; and/or
a modulator, a mutant or a nucleic acid according to the invention for use in treating or preventing a disease selected from the group consisting of an inflammatory disorder, an autoimmune disorder, a cardiovascular disease, a neurodegenerative disease, a graft versus host disease, a behavioral disorder, a cicatrisation disorder, a viral infection, cancer and pain.

More generally, the present invention is devoted to the generation of novel therapeutic compounds and drugs with increased efficacy and specificity for the treatment of mental or neurological diseases, or pathologies of the immune system, infectious diseases or cancer. These modulators could also be used as drugs for the treatment of pathological states for which specific therapeutic agents are now lacking. Published studies using a macaque model have already emphasized the potential of chemokine variants in topical drugs (Lederman, 2004, Science, 306, 485-487.). In the field of prevention of age-related handicaps, it is expected that surrogate agonists of an orphan GPCR responsible for the control of growth hormone release could revert some of the disabilities associated with ageing (Smith et al., 1999, Trends Endocrinol Metab. 10 (4), 128-135).

By “effective amount” is meant an amount sufficient to achieve a concentration of peptide which is capable of preventing, treating or slowing down the disease to be treated. Such concentrations can be routinely determined by those of skilled in the art. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like. It will also be appreciated by those of stalled in the art that the dosage may be dependent on the stability of the administered peptide.

By “individual in need thereof” is meant an individual suffering from or susceptible of suffering from the disease to be treated or prevented. The individuals to be treated in the frame of the invention are preferably human individuals. However, the veterinary use of modulators, mutants and nucleic acids for treating other mammals is also contemplated by the present invention.

By “treating” is meant a therapeutic use and by “preventing” is meant a prophylactic use.

In a preferred embodiment, the modulator, mutant or nucleic acid is or encodes an antagonist and said disease is selected from the group consisting of an inflammatory disorder, an autoimmune disorder, a cardiovascular disease, a neurodegenerative disease, cancer and a graft versus host disease. The inflammatory disorder may e.g. correspond to glomerulonephritis or lupus nephritis. Autoimmune disorders include, e.g., multiple sclerosis, rheumatoid arthritis, lupus erythematosus, inflammatory bowel disease and ulcerative colitis. Cardiovascular diseases include e.g. atherosclerosis, thrombosis, atherothrombosis and heart failure. Cancer include, e.g., breast cancer, colon cancer and lymphoma. The neurodegenerative disease may for example correspond to Parkinson's disease.

In another preferred embodiment, the modulator, mutant or nucleic acid is or encodes an agonist and said disease is selected from the group consisting of a viral infection and a behavioral disorder such as a disturbance of activity and attention. The viral infection may for example correspond to HIV infection. The behavioral disorder preferably corresponds to an attention deficit disorder, associated or not with hyperactivity.

The invention is also directed to:
a method for stimulating an anti-tumoral response or cicatrisation comprising the step of administering an effective amount of an agonist according to the invention, a mutant according to the invention having agonistic activity or a nucleic acid encoding an agonist according to the invention to an individual in need thereof; and/or
an agonist according to the invention, a mutant according to the invention having agonistic activity or a nucleic acid encoding an agonist according to the invention for use in stimulating an anti-tumoral response or cicatrisation.

The invention is further directed to:
a method for vaccinating an individual comprising the step of administering a vaccine composition comprising an effective amount of an agonist according to the invention, a mutant according to the invention having agonistic activity or a nucleic acid encoding an agonist according to the invention to said individual; and/or
an agonist according to the invention, a mutant according to the invention having agonistic activity or a nucleic acid encoding an agonist according to the invention for use in vaccination, and/or for use as an adjuvant in a vaccine composition.

The modulator, a mutant or a nucleic acid according to the invention may be administered through any route, preferably through the parenteral or the topical route.

Therapeutic Compositions Comprising Modulators, Mutants and Nucleic Acids in Accordance with the Invention

The modulators, mutants and nucleic acids described herein may be formulated into a pharmaceutical composition. Thus the invention contemplates a pharmaceutical composition comprising any one of the modulators, mutants and nucleic acids described herein and a physiologically acceptable carrier. Physiologically acceptable carriers can be prepared by any method known by those skilled in the art.

Pharmaceutical compositions comprising at least one modulator, mutant or nucleic acid of the invention include all compositions wherein the modulator, mutant or nucleic acid is contained in an amount effective to achieve the intended purpose. In addition, the pharmaceutical compositions may contain suitable physiologically acceptable carriers comprising excipients and auxiliaries which facilitate processing of the active compounds into preparations which can be used pharmaceutically. The term “physiologically acceptable carrier” is meant to encompass any carrier, which does not interfere with the effectiveness of the biological activity of the active ingredient and that is not toxic to the host to which is administered. Suitable physiologically acceptable carriers are well known in the art and are described for example in Remington's Pharmaceutical Sciences (Mack Publishing Company, Easton, USA, 1985), which is a standard reference text in this field. For example, for parenteral administration, the above active ingredients may be formulated in unit dosage form for injection in vehicles such as saline, dextrose solution, serum albumin and Ringer's solution. Besides the physiologically acceptable carrier, the compositions of the invention can also comprise minor amounts of additives, such as stabilizers, excipients, buffers and preservatives. The composition of the invention may further comprise a second active principle.

The modulators, mutants and nucleic acids of the present invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral and topical routes are particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Depending on the intended route of delivery, the compounds may be formulated as liquid (e.g., solutions, suspensions), solid (e.g., pills, tablets, suppositories) or semisolid (e.g., creams, gels) forms.

In a preferred embodiment, the physiologically acceptable carrier is a hydrogel matrix. The modulator, polypeptide or nucleic acid according to the invention is preferably covalently bound into the hydrogel matrix. Such hydrogel matrixes are very convenient for topical use, e.g. for enhancing and/or stimulating cicatrisation. Hydrogel matrixes are for example commercialized by Kuros Biosurgery AG (Zurich, Switzerland).

The invention also contemplates a pharmaceutical composition comprising a nucleic acid encoding the peptide of the invention in the frame of e.g. a treatment by gene therapy. In this case, the nucleic acid is preferably present on a vector, on which the sequence coding for the peptide is placed under the control of expression signals (e.g. a promoter, a terminator and/or an enhancer) allowing its expression. The vector may for example correspond to a viral vector such as an adenoviral or a lentiviral vector.

The invention further provides kits comprising a pharmaceutical composition comprising a modulator, mutant or a nucleic acid according to the invention and instructions regarding the mode of administration. These instructions may e.g. indicate the medical indication, the route of administration, the dosage, and/or the group of patients to be treated.

The invention also provides a pharmaceutical composition which is a vaccine, said vaccine comprising:
an immunogenic molecule;
a modulator, mutant or nucleic acid according to the invention; and
a physiologically acceptable carrier.

Such vaccines comprise an immunogenic molecule as the active principle and a modulator, polypeptide or nucleic acid according to the invention as an adjuvant. The role of the modulator, polypeptide or nucleic acid according to the invention is then to elicit the immune response to the immunogenic molecule. In the frame of this embodiment, the vaccine preferably comprises a nucleic acid according to the invention, wherein said nucleic acid encodes an agonist.

Methods of Producing Modulators and Mutants in Accordance with the Invention

The modulators and mutants of the invention may be produced by any well-known procedure in the art, including recombinant technologies and chemical synthesis technologies.

A preferred embodiment of the invention is directed to a method of producing a modulator or a mutant according to the invention comprising the step of:
a) providing a host cell comprising a nucleic acid according to the invention;
b) cultivating said host cell under conditions suitable for the expression of the modulator or mutant; and
c) isolating the modulator or mutant.

This method may further comprise the step of purifying said modulator or mutant, and optionally of formulating said modulator or mutant into a pharmaceutical composition.

In the frame of this embodiment, the nucleic acid according to the invention is preferably cloned into an expression vector. In such an expression vector, the nucleic acid of the invention is placed under the control of expression signals (e.g. a promoter, a terminator and/or an enhancer) allowing its expression.

In the frame of this embodiment, the nucleic acid according to the invention preferably encodes a polypeptide or mutant in accordance with the invention that comprises a leader sequence such as a signal peptide at its N-terminal extremity.

The host cell may correspond to any well-known host cell for protein production. Such host cells include human (e.g. 293, PER.C6), CHO, mouse, monkey, fungal (e.g. A. niger), yeast (e.g. S. cerevisiae) and bacterial (e.g. E. coli) cells.

All references cited herein, including journal articles or abstracts, published patent applications, issued patents or any other references, are entirely incorporated by reference herein, including all data, tables, figures and text presented in the cited references.

Although having distinct meanings, the terms “comprising”, “having”, “containing’ and “consisting of” have been used interchangeably throughout this specification and may be replaced with one another.

The invention will be further evaluated in view of the following examples and figures.

### BRIEF DESCRIPTION OF THE FIGURES

FIG. 1 depicts the strategy used to select phage-displayed CX3CR1 antagonists. Phage particles are represented as cylinders (encapsulated phage genome) with multiple heads (pIII-CX3CL1 mutant fusion). (1) The library of phage-displayed CX3CL1 mutants is allowed to bind HEK cells stably expressing the CX3CR1 receptor on the cell surface. (2) The cells were incubated at 37° C. to permit ligand-induced internalization of agonist phage particles while CX3CR1 antagonists do not enter into the cell. (3) Stringent washing was used to remove nonspecifically bound phage. (4) An excess of soluble CX3CL1 (sCX3CL1) was added to remove surface-associated phage. Eluted phages were allowed to infect E. coli, and stocks of selected phages were prepared to be used in a new round of selection (steps 1 to 4).

FIG. 4 shows the chemotactic activity of F1 and F1-Ig. Both chemokine and chemokine-Ig were tested for their chemotactic potency on CD8+ T cells (A) NK cells (B) and CD4+ T cells (C). Results are expressed as a chemotaxis index. D. F1 dose-dependently inhibits the chemotaxis of CD8+ T cells (filled diamonds) and NK cells (empty diamonds) induced by 1 nM CX3CL1. Data are fitted with a standard dose-response curve (GraphPad Prism software). The IC50 was 6.6 nM (LogIC50=0.82±0.8 nM) for CD8+ T cells and 2.9 nM (LogIC50=0.46±0.3 nM) for NK cells.

FIG. 3 shows a Calcium assay of F1. A. HEK-CX3CR1 cells (traces a and b) or PBMC (traces c and d) were tested for calcium response to 100 nM of CX3CL1 (traces a and c) or to F1 at the indicated concentration (traces b and d). Shown is one representative experiment of three. B. The inhibitory activity of F1 (filled triangles) or F1-Ig (empty triangles) on calcium response elicited in PBMC by 20 nM CX3CL1 was assayed. Data (triplicates±SD) are fitted with a standard dose-response curve (GraphPad Prism software). The IC50 was 34 nM (LogIC50=1.53±0.08 nM) for F1 and 72 nM (LogIC50=1.86±0.14 nM) for F1-Ig.

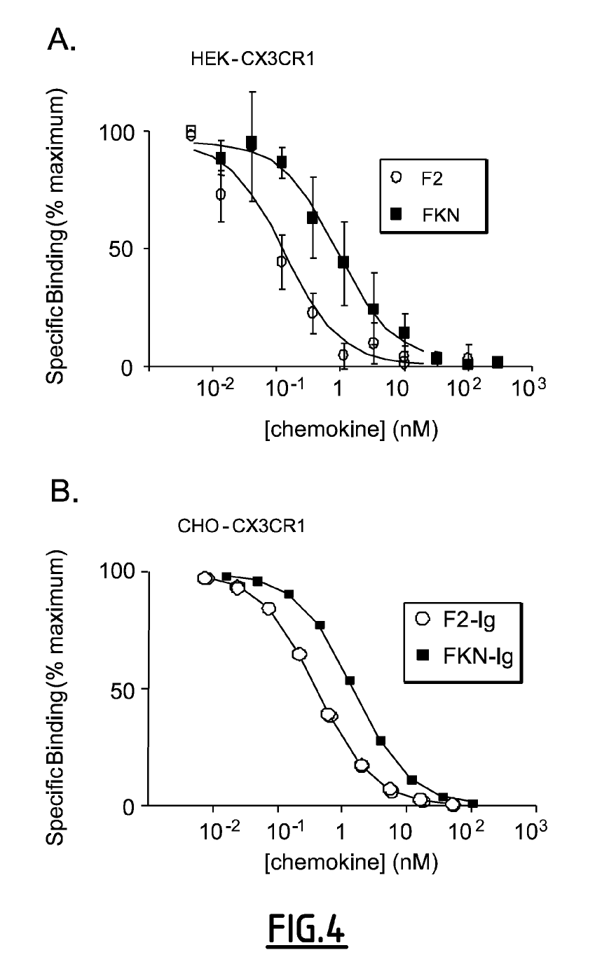

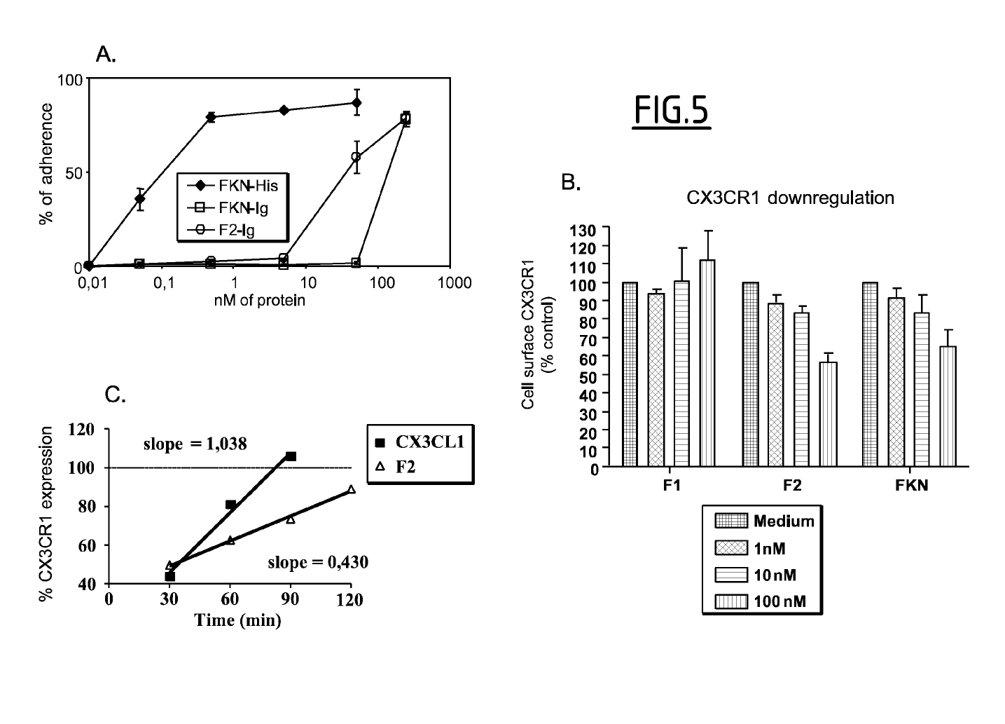

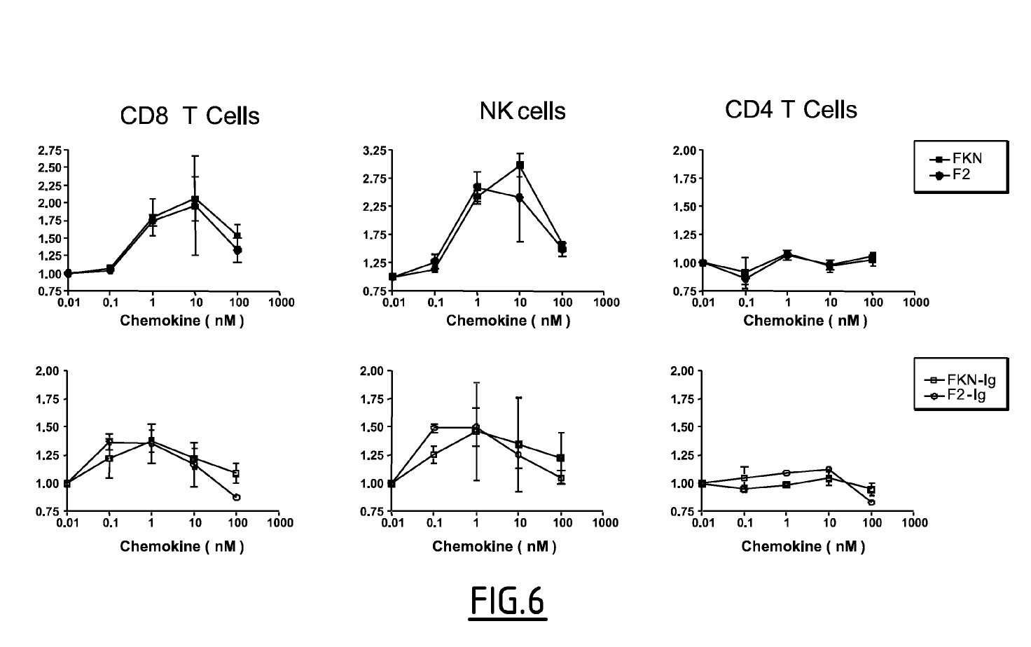

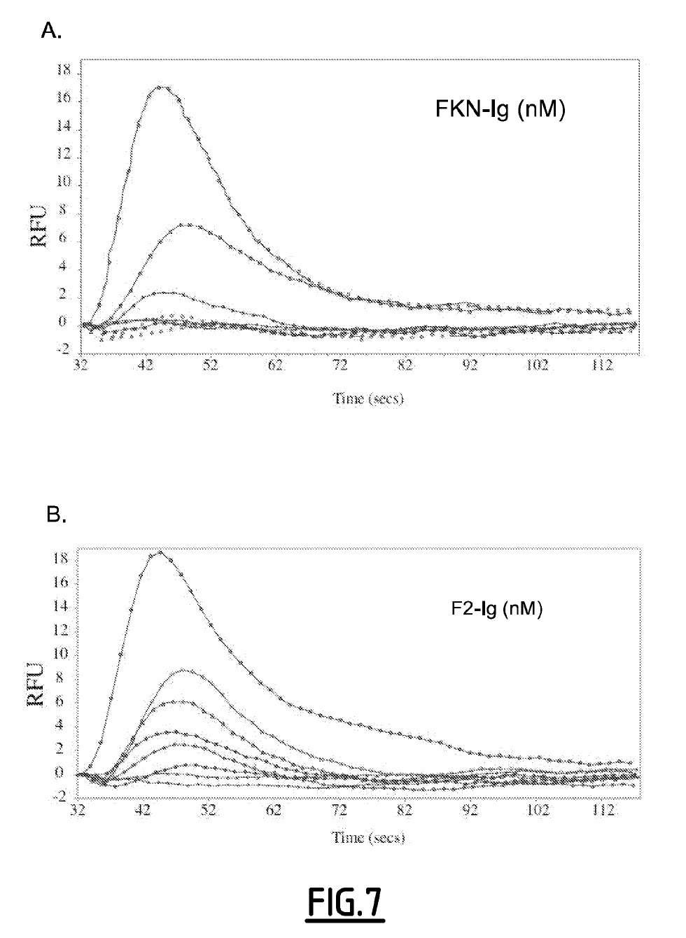

FIG. 4 shows the binding of F2 and F2-Ig to human CX3CR1, as measured by competition binding assay on CX3CR1 stably expressing cells. A. Binding on CX3CR1-expressing HEK cells. B. Binding on CX3CR1-expressing CHO cells.

FIG. 5 shows some characteristics of F2 and/or F2-Ig. A. Adherence of HEK-CX3CR1 to immobilized CX3CL1-His, CX3CL1-Ig and F2-Ig. Data are expressed in percentage of maximum of adherence in each condition. B. Downregulation of CX3CR1 from the surface of stably transfected HEK cells. HEK-CX3CR1 cells were incubated for 30 min at 37° C. with CX3CL1 or CX3CL1 variants. Surface CX3CR1 was detected with a monoclonal CX3CR1 antibody and analyzed by flow cytometry. CX3CL1 and F2 induced dose dependant receptor down-modulation while no downregulation was observed with F1. C. Recycling of CX3CR1 on HEK-CX3CR1 after downregulation with CX3CL1 (open triangles) and F2 (black squares). Cells were first incubated for 30 min at 37° C. with 100 nM of chemokine. After several washes, cells were further cultured in medium at 37° C. for various periods of time and analyzed for CX3CR1 expression.

FIG. 6 shows the chemotactic activity of F2 (upper line) and F2-Ig (lower line). Both F2 and F2-Ig were tested for their chemotactic potency on CD8+ T cells (left column), NK cells (center column) and CD4+ T cells (right column). Results are expressed in chemotaxis index, said index representing the number of cells migrating in response to chemokine relative to the number of cells migrating in the absence of chemokine.

FIG. 7 shows the results of a calcium assay of F2-Ig on CX3CR1 expressing cells. Cytosolic calcium-dependent fluorescence changes in response to various concentrations of FKN-Ig and F2-Ig (0, 0.2, 0.6, 1.9, 5.5, 16.7, 50 and 150 nM) were determined on CHO-CX3CR1 cells that had been loaded with Fluo-4 dye.

### BRIEF DESCRIPTION OF THE SEQUENCES

SEQ ID Nos. 1-7 and 64 correspond to consensus sequences of modulators according to the invention.

SEQ ID Nos. 8-24 correspond to the sequences of antagonists identified as described in Example 2.

SEQ ID Nos. 25-51 correspond to the sequences of agonists identified as described in Example 2.

SEQ ID NO: 52 corresponds to the six N-terminal amino acids of mature human CX3CL1.

SEQ ID NO: 53 corresponds to the six N-terminal amino acids of mature rat and murine CX3CL1.

SEQ ID NO: 54 corresponds to the sequence of human CX3CL1 (before proteolytic processing).

SEQ ID Nos. 55 and 56 correspond to mutants of a human CX3CL1 polypeptide.

SEQ ID NO: 57 corresponds to a mutant of a human CX3CL1 polypeptide fused to a domain of an immunoglobulin.

SEQ ID NO: 58 corresponds to the nucleic acid sequence of the CDS of CX3CL1.

SEQ ID Nos. 59-62, 69 and 70 correspond to oligonucleotides used in Example 1.

SEQ ID NO: 63 corresponds to the sequence of human CX3CR1.

SEQ ID NO: 64 corresponds to a sequence used in the frame of Example 2.

SEQ ID Nos. 65 and 66 correspond to the nucleotidic and polypeptidic sequences of a mutant of a human CX3CL1 polypeptide.

SEQ ID Nos. 67 and 68 correspond to the nucleotidic and polypeptidic sequences of a mutant of a human CX3CL1 polypeptide, fused to a domain of an immunoglobulin.

### EXAMPLES

### Example 1

### Protocols

1.1. Cell Lines

Human monocytic leukemia (THP-1), human embryonic kidney (HEK) and Chinese hamster ovary (CHO and CHO-S) cell lines were routinely maintained in DMEM supplemented with 2 mM L-glutamine, 1% (v/v) nonessential amino acids, 2 mM sodium pyruvate, 10% FBS, penicillin (50 U/mL) and streptomycin (50 μg/mL). HEK-CCR5 and HEK-CX3CR1 cells have been described by Combadiere et al. (1996, J. Leukoc. Biol. 60, 147-152) and Combadiere et al. (1998, J. Biol. Chem. 273, 23799-23804). The CHO cells expressing human CX3CR1 were a gift from Dr. Jeffrey K. Harrison (Department of Pharmacology and Therapeutics, College of Medicine, University of Florida, Gainesville, Fla., USA). Human peripheral blood mononuclear cells (PBMC), obtained from healthy donors, and mouse mononuclear bone marrow cells (MBMC) from C57BL/6 mice were purified with Ficoll-Hypaque gradient centrifugation.

1.2. Phage-Chemokine and Libraries Constructions

The DNA sequence coding for human CX3CL1 was amplified by PCR from pBlast-hCX3CL1 plasmid (Invivogen, San Diego, Calif., USA) with Nco I-tailed forward primer 5′-CCGGCCATGGCCCAGCACCACGGTGTGAC (SEQ ID NO: 59) and Not I-tailed reverse primer 5′ TTGTTCTGCGGCCGCGCCATTTCGAGTTAG (SEQ ID NO: 60) (the recognition sites for endonuclease are underlined). The PCR product was cut and sub-cloned into a pHEN1 phagemid vector as described by Hoogenboom et al. (1991, Nucleic Acids Res 19, 4133-7). The library of N-terminal CX3CL1 mutants was constructed by PCR mutagenesis, essentially as reported by Hartley et al. (2003, J Virol 77, 6637-44) with the Not I-tailed reverse primer and degenerate upstream primers 5′-CCGGCCATGGCCNNKCNANNKNNKGNCNTGNCAAAATGCAACATCACGTGC (SEQ ID NO: 61) and 5′-CCGGCCATGGCCNNKNNKNNKGNCNTGNCAAAATGCAACATCACGTGC (SEQ ID NO: 69). The recognition site for Nco I is underlined, N represents any of the four bases, K represents either G or T. The PCR products were cloned into phagemid pHEN1 cut Nco I-Not I and electroporated into E. coli TG1. Colonies were PCR-screened before selection, and their DNA inserts were sequenced with an automatic sequencer ABI 377 (Applied Biosystems, Perkin-Elmer, Waltham Mass. USA) to check the library diversity.

1.3. Selection of CX3CR1 Antagonists on Living Cells

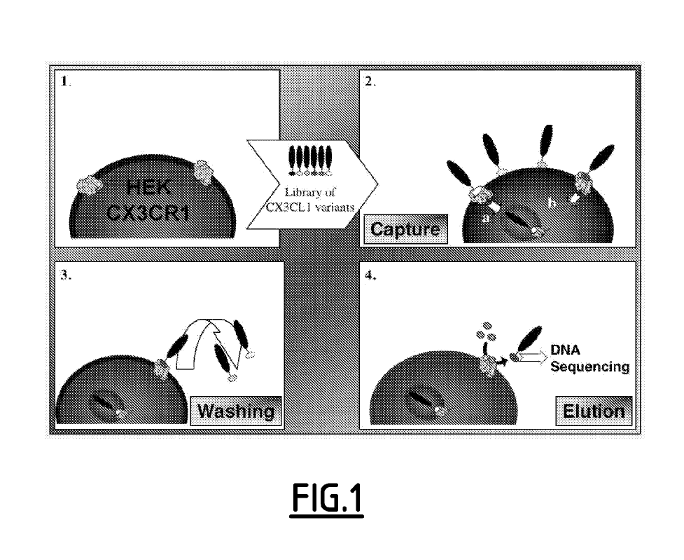

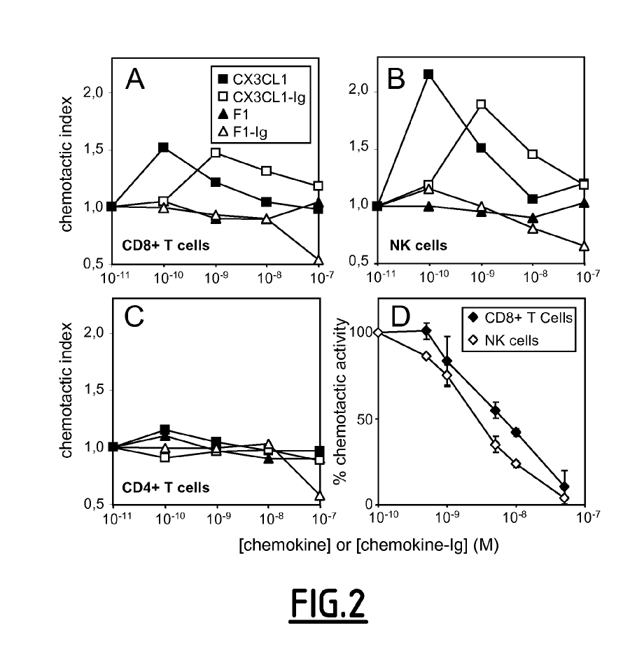

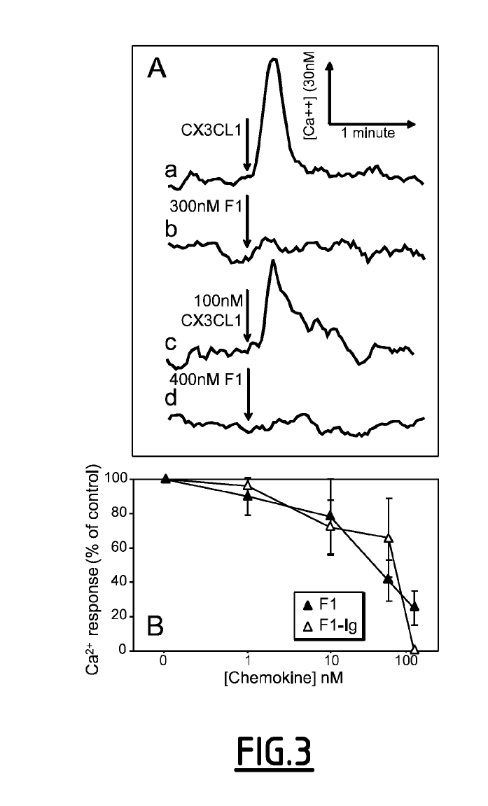

The selection strategy is presented in FIG. 1. A phage library of CX3CL1 mutants (10¹⁰ CFU) was directly incubated with 5.10⁶ HEK-CX3CR1 or HEK-CCR5 cells growing in 25 cm² tissue culture flasks (Becton Dickinson, Le Pont de Claix, France) at 37° C. at 5% CO₂ in 5 ml of supplemented RPMI-1640 medium. After 1 h, cells were washed 10 times at room temperature with 10 ml of phosphate-buffered saline (PBS) and then scraped from the plate into 10 ml of PBS-0.5% BSA. Cells were then pelleted and incubated for 20 min on ice in elution buffer consisting of an excess of soluble CX3CL1 (10 μM in 100 μl PBS-BSA). Cells were centrifuged (1000×g for 5 min) and supernatant was mixed with log-phase E. coli TG1 for production and purification of stock phage to be used in further rounds of selection, as described by Hartley et al. (2003, J Virol 77, 6637-44).

1.4. Selection of CX3CR1 Agonists on Living Cells

The selection strategy was the same as the one described in paragraph 1.3. herabove, except that phages that had been endocytosed by the cells were recovered. The method used for selecting agonists is described in Hartley et al. (2003, J Virol 77, 6637-442003).

1.5. Phage Display of Chemokine Domain

The purified phage-chemokines were used in phage-ELISA as described by Dorgham et al. (2005, AIDS Res Hum Retroviruses 21, 82-92) with the anti-human CX3CL1 polyclonal antibody (AF365, R&D, Lille, France) as coating. For flow cytometry analysis, phages (10¹⁰ CFU/ml) were incubated at 4° C. with 10⁵ HEK or HEK-CX3CR1 cells. Cells were washed and incubated with anti-M13 antibody (Pharmacia, Saclay, France) labeled with FITC as described by Sambrook et al. (1989, Molecular Cloning. A Laboratory Manual. Cold Spring Harbor Laboratory, Cold Spring Harbor) and analyzed on FACSCalibur (BD Bioscience, Le Pont de Claix, France) with Cell Quest software.

1.6. Preparation of CX3CL1 Analogue and Chimeric Immunoglobulin Fusion Chemokines

F1 and F2, which are CX3CL1 analogues, were prepared by total chemical synthesis, essentially as described by Hartley et al. (2004, Proc Natl Acad Sci USA 101, 16460-5). Compound purity and integrity was verified by analytical high-performance liquid chromatography and mass spectrometry. Concentrations were determined by measurement of absorbance at 280 nm.

The chimeric chemokine-Ig construct was prepared as previously described by Layergne et al. (2003, Cancer Res 63, 7468-74) and by Iga et al. (2007 Vaccine 25, 4554-63). The murine Fcγ2a fragment mutated in the Clq binding motif (E318, K320, K322) and the FcγRI binding site (L235) (Altman et al. 1996, Science 274, 94-96; Zheng et al. 1995, J Immunol 154, 5590-600) was used to produce a noncytolytic form of F1-Ig and F2-Ig. The DNA sequence of F1 and F2 were amplified from the phage display vector with the Not I-tailed reverse primer and a specific upstream primer encoding the human CX3CL1 signal peptide 5′-AAAACTGCAGCCATGGCTCCGATATCTCTGTCGTGGCTGCTCCGCTTGGCCACCTTCTGCCATCTGACTGTCCTGCTGGCTGGAATTCTAGATAATGGCGTGTCA-3′ (SEQ ID NO: 62) and 5′-AAAACTGCAGCCATGGCTCCGATATCTCTGTCGTGGCTGCTCCGCTTGGCCACCTTCTGCCATCTGACTGTCCTGCTGGCTGGACAGCCTCAGGGCGTGTCAAAA-3′ respectively (SEQ ID NO: 70). The recognition site for Pst I is underlined. The PCR product was cloned into a pVRC vector (Layergne et al. 2003, Cancer Res 63, 7468-74; Layergne et al. 2004, J Immunol 173, 3755-62).

Low-endotoxin pVRC chemokine-Ig (5 μg) and empty pBlast plasmids (1 μg) were co-transfected into a CHO-S cell line (Invitrogen, Cergy-Pontoise, France) with JetPEI™ transfection reagent according to manufacturer's instruction (Polyplus-transfection SA, Illkirch, France). Transfectants were selected by adding 10 μg/ml blasticidin (Invivogen Cayla, Toulouse, France) and maintained with 5 μg/ml blasticidin. High-producing clones were selected by screening supernatants for CX3CL1 by capture ELISA (Human CX3CL1/Fractalkine, R&D, Lille, France). Chemokine-Ig fusion proteins from 500 to 1000 ml of culture supernatant were purified through protein G columns (NUNC ProPur Kit Midi G, VWR International S.A.S. Fontenay sous Bois, France). The protein was buffer-exchanged and concentrated to a final volume of 1 mL in PBS. Chimeric protein solutions were tested by SDS-PAGE, silver staining, and immunoblotting assays to estimate purity of preparations. Protein concentrations in solution were determined by measurement of absorbance at 280 nm and capture ELISA before the in vivo experiments.

1.7. CX3CR1 Receptor Down-Modulation Experiments

Cells (10⁵) were incubated for 30 min at 37° C. in 100 μl of supplemented culture medium containing various concentrations (1 to 1000 nM) of chemokines. Medium alone was used as a control. Cells were washed 5 times with cold PBS and incubated for 30 min on ice with 50 ng of anti-human CX3CR1 mAb (clone 2A9-1 phycoerythrin-conjugated, MBL Clinisciences, Montrouge, France). Cells were washed twice, fixed with 4% paraformaldehyde, and analyzed with a FACScalibur and Cell Quest software. At least 10,000 events were accumulated for each sample. The percentage of surface CX3CR1 expression was calculated according to the mean channels of relative fluorescence intensity (MCF) as follows: (MCF chemokine−MCF negative control)/(MCF medium−MCF negative control) (Mack et al. 1998, J Exp Med 187, 1215-24).

1.8. Competitive Radioligand Binding to CX3CR1

Assays were performed as previously described by Moatti et al. (2001, Blood 97, 1925-8). Competitive binding to HEK-CX3CR1 and CHO-CX3CR1 cells was performed with 50 pM [¹²⁵I]-CX3CL1 (Amersham General Electric, Saclay, France) plus variable amounts of unlabeled ligand. Each concentration was assayed in duplicate. After 2 h at 37° C., cells were washed and radioactivity in the cell pellet was quantified with a gamma counter (LKB Wallac, Saint Quentin en Yvelines, France).

1.9. Calcium Mobilization Assay

Cytosolic free calcium was measured with Fura-2/AM (Molecular Probes, Leiden, Netherlands), essentially as described by Garin et al. (2003, J Immunol 171, 5305-12). Briefly, PBMC (4×10⁶) were loaded for 30 min at 37° C. with 2 μM Fura-2/AM and 2 μM pluronic acid in 1 ml of HBSS buffer supplemented with 10 mM HEPES, 0.5 mM MgCl₂, and 1 mM CaCl₂. Cells were centrifuged and transferred to quartz cuvettes for reading. Chemokines and chemokine-Ig fusion proteins were added to the cells at various concentrations in cuvettes thermostatically maintained at 37° C. and stirred continuously. Fluorescence was monitored with a spectrofluorometer (SAFAS, Monaco) at 340 and 380 nm and measured at 510 nm.

1.10. Chemotaxis Assay

Migration assays were performed in 24-transwell inserts (Corning Costar, Avon, France) with 5-μm pore polycarbonate filters for human PBMC and 8-μm filters for mouse MBMC. Cells were resuspended in chemotaxis buffer (5.10⁵ cells in 100 μl RPMI containing 0.5% BSA and 10 mM HEPES) and loaded into the top chamber. The bottom of each well was filled with 600 μl of prewarmed chemotaxis buffer with the indicated chemokine concentration. The plates were then incubated for 3 h at 37° C. in a 5% CO₂ atmosphere. Cells that passed through the membrane were immunophenotyped by mixing fluorescent antibodies (anti-human CD45-FITC, CD8-APC, CD3-PE or anti-mouse CD11b-FITC, BD, Le Pont de Claix, France) and a predetermined number of beads (Flow-Count™ Fluorospheres, Beckman Coulter, Villepinte, France). After 30 min incubation in ice, beads and cells were counted on a FACSCalibur flow cytometer, and data were analyzed with Cell Quest software. Results are expressed as a chemotaxis index (CI) that represents the ratio of cells migrating in the presence versus the absence of chemoattractant. All conditions were run in duplicate and results are representative of at least three independent experiments.

1.11. Adhesion Assay

CX3CL1-H₆ (R&D Systems, Lille, France, 1 nM in 50 μL per well) or the purified chemokine-Ig proteins (diluted at the indicated amount) were plated overnight at 4° C. on Maxisorb 96-well microtiter plates (Nunc A/S, Roskilde, Denmark) in buffer containing 25 mM Tris, pH 8, 150 mM NaCl. HEK-CX3CR1 cells loaded with CFDA-SE (Invitrogen, Cergy-Pontoise, France) were incubated for 45 min at room temperature in the presence or absence of CX3CL1 or F1 at the indicated concentrations in plates previously blocked with 1% nonfat milk. To remove nonadherent cells, the wells were gently filled with PBS and the microplate was placed floating upside down in PBS for 1 h before reading at 535 nm with a Fusion Universal Microplate Analyzer (Packard Bioscience, Perkin Elmer, Villebon sur Yvette, France), as described by Hermand et al. (2000, J Biol Chem 275, 26002-10). Experiments were performed in triplicate and results expressed as the percentage of total adherent cells (±SD).

1.12. Inflammation Induced by Thioglycollate

Wild-type 6-10 week-old female C57BL/6 mice (Janvier, Le Genest Saint Isle, France) were injected intraperitoneally with 1 ml 3% (wt/vol) thioglycollate (Sigma-Aldrich, I'lle d'Abeau, France) dissolved in sterile PBS and 14 hours later with 50 μl of 500 nM chemokine analogue or PBS. Two days later, the mice were killed and 3 ml of cold PBS was injected intraperitoneally to recover peritoneal cells, which were then stained with anti-CD11b-FITC, anti-Ly6G-PE and anti-7/4-APC (R&D Systems, Lille, France). At least 7.10⁶ cells per mouse were counted. The percentages and absolute numbers of different cell types were calculated. The local animal experimentation and ethics committee approved the experimental protocol.

1.13. CX3CR1 Recycling

To study recycling of CX3CR1, HEK CX3CR1 cells were first incubated for 30 min at 37° C. with 100 nM FKN(CXCL1), 100 nM F2, or medium as control. Cells were washed four times in medium at room temperature and further incubated at 37° C. Aliquots were taken at various times, stained, and analyzed as described above in paragraph 1.13. Linear regression was analyzed and slopes calculated using GraphPad Software.

### Example 2

### Engineering the CX3CR1 Modulators

2.1. Engineering the CX3CR1 Antagonists

Phage particles can be efficiently endocytosed by mammalian cells in a receptor-dependent manner, and phage-chemokine agonists can be recovered after cell lysis. A modified phage display-based selection strategy with live-cell competitive elution was used to select preferentially for CX3CL1 variants with antagonist properties. In this strategy, the phage library was incubated with CX3CR1-expressing cells at 37° C. to allow ligand-induced internalization of agonist phage particles. Phage displaying CX3CR1 antagonists would not enter the cell and would therefore be susceptible to competitive elution with a large excess of soluble CX3CL1.

The human CX3CL1 chemokine domain, which consists of the first 77 residues of the mature protein, was cloned for expression by phage display. CX3CL1-phage showed detectable binding on anti-CX3CL1 antibody but not isotype control antibody. Additionally, CX3CL1-phage bound to HEK cells that expressed CX3CR1 but not to either parental HEK or CCR5-expressing cells. CCL5-expressing phage did not bind to either the anti-CX3CL1 antibody or the HEK-CX3CR1 cells. These results show that CX3CL1-phage bound specifically to CX3CR1-expressing cells.

Based on our previous work and on our evidence that CX3CL1 is successfully expressed on phage, PCR mutagenesis was used to design and express a phage library of CX3CL1 variants, where the first six residues of the CX3CL1 DNA sequence were completely or partially randomized. In another phage library of CX3CL1 variants, a one-residue N-terminal extension (position 0) was added into our library design, Finally, the two libraries of CX3CL1 mutants had the following composition:
X₀Z₁X₂X₃Σ₄φ₅ψ₆-CX3CL1(7-76) (i.e. SEQ ID NO: 64 fused to amino acids 31 to 100 of SEQ ID NO: 54); and
X₁X₂X₃Σ₄φ₅ψ₆-CX3CL1(7-76) (i.e. SEQ ID NO: 7 fused to amino acids 31 to 100 of SEQ ID NO: 54)

where X is any amino acid, Z is L, P, Q or R; Σ is V, A, D or G; φ is L, M or V and ψ is S, P, T or A (Table 1).

After four rounds of selection on HEK-CX3CR1 cells, the antagonists shown in table 1 herebelow were identified.

TABLE 1

| | Number of | |
|---|---|---|
| N-terminal | isolated | SEQ ID |
| sequence | sequences | NO: |
| ILDNGVS | 15 | 8 |
| TLAQGLP | 5 | 9 |
| ILDGGVS | 3 | 10 |
| FLQSDVA | 1 | 11 |
| ILDLGLS | 1 | 12 |
| ILDLGLT | 1 | 13 |
| ILDNGVA | 1 | 14 |
| ILGRDVA | 1 | 15 |
| QPLFAVA | 1 | 16 |
| QRDSVLA | 1 | 17 |
| SLDHGLS | 1 | 18 |
| SLIPVVP | 1 | 19 |
| TLPQGLA | 1 | 20 |
| WLSQGLA | 1 | 21 |
| QSLVLP | 1 | 22 |
| QLFALS | 1 | 23 |
| VQSVLS | 1 | 24 |

A comparison of the selected sequences allowed the definition of a consensus sequence: ILDXGL/VA/S (SEQ ID NO: 6), where X is any amino acid.

To ascertain whether the selection of this consensus sequence was CX3CR1-specific, the same selection procedure was performed on HEK-CCR5 cells. After two rounds of biopanning, most of the selected phage had lost the CX3CL1 gene insert.

Finally, it was confirmed that a preferentially selected phage clone, ILDNGVS (SEQ ID NO: 8), henceforth called F1, bound to an immobilized anti-CX3CL1 antibody and to CX3CR1-expressing cells. This binding was specific: phage-F1 did not recognize control IgG or parental HEK or HEK-CCR5.

For further analysis, chemical synthesis was used to produce the F1 analogue as a soluble protein, corresponding to the CX3CL1 chemokine domain. In addition, a fusion protein of F1 with the murine Fc fragment of immunoglobulin (F1-Ig) was constructed to generate a variant of F1 with a longer in vivo half-life.

2.2. Engineering the CX3CR1 Agonists

CX3CR1 agonists were isolated by recovering endocytosed phages. The same libraries as described in paragraph 2.1. hereabove were used. After rounds of selection on HEK-CX3CR1 cells, the agonists shown in table 2 herebelow were identified.

TABLE 2

| | Number of | |
|---|---|---|
| N-terminal | isolated | SEQ ID |
| sequence | sequences | NO: |
| QPGGVS | 9 | 25 |
| QPQAVS | 5 | 26 |
| QPVALA | 5 | 27 |
| QPVGLS | 5 | 28 |
| AAQGMS | 4 | 29 |
| QPGAVS | 4 | 30 |
| QPMGVA | 4 | 31 |
| QPQGLA | 4 | 32 |
| QPVAVA | 4 | 33 |
| QHLGLS | 3 | 34 |
| QLQGLA | 3 | 35 |
| QPSALS | 3 | 36 |
| QSLGVS | 3 | 37 |
| GPQAMS | 2 | 38 |
| NPQALS | 2 | 39 |
| QFPGVS | 2 | 40 |
| QLLGVS | 2 | 41 |
| QPHGVA | 2 | 42 |
| QPRALP | 2 | 43 |
| QPSALT | 2 | 44 |
| QPSGMS | 2 | 45 |
| QPVAVS | 2 | 46 |
| QPYGMS | 2 | 47 |
| QPYGVS | 2 | 48 |
| QSPGMS | 2 | 49 |
| QVQGVT | 2 | 50 |

These selected sequences allowed identifying the QPQGVS (SEQ ID NO: 51) consensus sequence.

### Example 3

### Characterization of F1 as CX3CR1 Ligand

The binding affinity of F1 for CX3CR1 was compared to that of native

CX3CL1 in a competition binding, with HEK-CX3CR1 cells and [¹²⁵I]-CX3CL1 as a tracer. The F1 analogue interacted with CX3CR1, although with a lower affinity than CX3CL1. An apparent binding affinity (IC₅₀) of 1.9 nM (Log IC₅₀=−8.73±0.21; n=3) was determined for F1, which is approximately 12 times weaker than that of CX3CL1 (IC₅₀=0.16 nM; Log IC₅₀=−9.79±0.28; n=3). A similar difference in affinity was apparent when F1 and CX3CL1 were tested on CHO-CX3CR1 cells, and when the affinity of CX3CL1-Ig was compared to that of F1-Ig. Together, these data confirm that the F1 analogue binds specifically to CX3CR1, albeit with a slightly weaker affinity than native CX3CL1.

The phage selection strategy for antagonists was devised to preferentially select clones that are not internalized into CX3CR1-expressing cells. It was confirmed that, unlike native CX3CL1, which induced dose-dependent down-modulation of CX3CR1, F1 did not induce CX3CR1 internalization on HEK cells: while 0.1 μM of CX3CL1 induced 40% CX3CR1 internalization after 30 min incubation, 1 μM of F1 had no internalizing effect. Up-regulating activity was even observed, as reported with CCR5 antagonist. A similar result was obtained with CHO-CX3CR1 cells. This might be due to marginal activation of CX3CR1 by traces of CX3CL1 in the culture medium or by the receptor's own intrinsic activity, which could be inhibited by F1, here functioning as an inverse agonist.

### Example 4

### F1 Antagonizes CX3CL1-Induced Calcium and Chemotactic Responses

The ability of F1 to elicit calcium response in HEK-CX3CR1 cells was then compared to that of CX3CL1. In contrast to native CX3CL1, no significant response to F1 was observed up to concentrations of 300 nM in HEK-CX3CR1 cells (FIG. 3A, compare traces a and b) or 400 nM in human PBMC (FIG. 3A, compare traces c and d) and in CHO-CX3CR1 cells. Similar results were obtained with the F1-Ig chimera. The ability of F1 to inhibit CX3CL1-induced cellular responses was next tested. In the presence of both F1 and F1-Ig, the calcium response induced by 20 nM CX3CL1 decreased dose-dependently (FIG. 3B), with an IC₅₀ of 34 nM and 72 nM respectively.

The effect of F1 on CX3CR1-mediated chemotaxis was then investigated (FIG. 2). CX3CL1 elicited significant responses on CD8+ T cells and NK cells, and CX3CL1-Ig induced chemotaxis with full efficacy but reduced potency, consistently with its binding affinity. In contrast, neither F1 nor F1-Ig induced chemotaxis at any concentration tested. None of the ligands tested induced any detectable chemotaxis by CD4+ T cells (FIG. 2C), which, except for the minute Th1 cytotoxic subpopulation, do not express CX3CR1.

Both F1 and F1-Ig were capable of inhibiting CX3CL1- and CX3CL1-Ig-induced chemotaxis of NK cells and of CD8+ T cells (FIG. 2D and Table 3 herebelow) in a dose-dependent manner, with IC₅₀ values of 2.7 nM and 6.1 nM respectively (Log IC₅₀=0.44±0.07; n=3 and 0.79±0.17; n=3). Similar results were obtained with human THP-1 and murine CD11b+MBMC (Table 3).

TABLE 3
Chemotactic activity of CX3CL1 and its analogues
on human and murine cells

| | | | | |
|---|---|---|---|---|
| Chemotaxis | | | HEK- | |
| (CI) | CD8 T cells | NK cells | CX3CR1 | MBMC CD11b |

A. CX3CL1 and F1 chemokines

| | | | | |
|---|---|---|---|---|
| 1 nM CX3CL1 | 1.53 | 2.20 | 1.33 | 1.16 |
| 1 nM F1 | 0.90 | 0.97 | 0.90 | 1.07 |
| 10 nM F1 | 0.89 | 0.90 | 0.93 | 0.91 |
| Chemotaxis | 100 | 100 | 100 | 100 |
| with of 1 nM | | | | |
| CX3CL1 | | | | |
| (% of control) | | | | |
| +1 nM F1 | 82 | 74 | ND | 40 |
| +10 nM F1 | 51 | 27 | ND | 27 |

B. CX3CL1-Ig and F1-Ig chimeric chemokine

| | | | | |
|---|---|---|---|---|
| 10 nM | 1.47 | 2.06 | 1.47 | 1.35 |
| CX3CL1-Ig | | | | |
| 1 nM F1-Ig | 0.89 | 0.97 | 0.98 | 1.00 |
| 10 nM F1-Ig | 0.87 | 0.90 | 0.85 | 1.08 |
| Chemotaxis | 100 | 100 | 100 | 100 |
| with | | | | |
| of 10 nM | | | | |
| CX3CL1-Ig | | | | |
| (% of control) | | | | |
| +1 nM F1-Ig | 76 | 66 | 53 | 53 |
| +10 nM F1-Ig | 67 | 31 | 10 | 22 |

Hence F1 is an efficient antagonist of both human and murine CX3CR1 receptors, both as a soluble chemokine domain and as an Ig-fusion protein.

### Example 5

### F1 Antagonizes CX3CL1-Mediated Adhesion

The complete CX3CL1 molecule, comprising the chemokine domain linked to the cell surface via a mucin stalk, has substantial adhesive properties when paired with its receptor CX3CR1. Because the adhesiveness of CX3CL1 is reported to be independent of the nature of the stalk, it was hypothesized that CX3CL1-Ig might behave as an adhesion molecule.

In a static adhesion assay, CX3CL1-Ig significantly captured HEK-CX3CR1 cells, while nonspecific Ig did not. Moreover, parental HEK cells did not adhere to CX3CL1-Ig. F1-Ig also specifically captured CX3CR1-expressing cells, albeit with an apparent potency one eighth that of CX3CL1-Ig, consistent with its lower binding affinity.

Moreover, soluble F1 substantially decreased the adhesion of CX3CR1-positive cells to immobilized CX3CL1: adhesion efficacy was 40 times lower than that of soluble CX3CL1. Thus, F1 also antagonized the CX3CL1/CX3CR1-mediated cell adhesion. These results also indicate that the antagonist potency of F1 differs slightly in binding and in adhesion assays.

### Example 6

### F1 Acts as an In Vivo CX3CR1 Antagonist

The in vivo inhibitory action of F1 was further evaluated with the thioglycollate-induced peritonitis model Boring et al. (1997, J Clin Invest 100, 2552-61).

Sixty two hours after intraperitoneal injection of thioglycollate, circulating mononuclear cells recruited into the peritoneal cavity were analyzed by flow cytometry for their expression of CD11b, Ly6G and 7/4. Monocyte (CD11b+Ly6G-7/4+) recruitment decreased significantly in mice treated with one injection of F1 14 hours after thioglycollate injection. In contrast, migration of the PMN population (CD11b+Ly6G+), which is CX3CR1-negative, was not significantly affected by F1 administration. The decreased monocyte recruitment was particularly apparent in the 7/4^{lo} sub-population (FIG. 6C), which expressed high levels of CX3CR1, while the migration of 7/4^{hi} monocytes) (CX3CR1^{lo} was almost unaffected.

### Example 7

### Conclusion

In this study, a phage display strategy was used to select both agonistic and antagonistic CX3CL1 chemokine analogues. Agonistic ligands are able to bind the CX3CR1 receptor and enhance CX3CR1 signaling. In contrast to this, antagonistic ligands are able to bind the CX3CR1 receptor without causing agonist-induced signaling. Several such agonistic and antagonistic CX3CL1 analogues were selected (SEQ ID Nos. 7-50). These selected CX3CL1 analogues allowed defining consensus sequences for CX3CR1 modulators (SEQ ID Nos. 1-6 and 51).

The F1 antagonistic analogue was further characterized. F1 did not induce any CX3CR1 internalization or any calcium (FIG. 3A) or chemotactic (FIGS. 2A and 2B) responses. Moreover, F1 inhibited CX3CL1-induced calcium responses (FIG. 3B), as well as the chemotactic (FIG. 2D) and adhesive functions mediated by the CX3CL1-CX3CR1 axis. Finally, F1 promoted significant inhibition of monocyte recruitment during an in vivo inflammation test. It therefore represents a bona fide antagonist of human CX3CR1.

Like F1, most of the selected chemokine antagonists bore the N-terminal consensus motif ILD, corresponding to residues 0, 1, and 2 of the native protein (residue 0 representing an N-terminal extension) (Table 1). This motif is more hydrophobic than the QHHGVT sequence of the native CX3CL1, due to the presence of the aliphatic I and L residues, and also more acidic (D versus H). On the other hand, there was no obvious selection at position 3 and the residues selected at positions 4-6 resembled those found in human and murine CX3CL1 (Table 1). It is therefore likely that the ILD motif at the extended N-terminus of the protein plays a key role in the antagonistic properties of F1.

Most of the selected chemokine antagonists were derived from the phage library comprising an additional one-residue N-terminal extension (position 0) compared to the N-terminal extremity of the native mature CX3CL1 protein (i.e. library X₀Z₁X₂X₃Σ₄φ₅ψ₆-CX3CL1(7-76)). In contrast to this, all the selected chemokine agonists were derived from the X₁X₂X₃Σ₄φ₅ψ₆-CX3CL1(7-76) library.

The F1 analogue antagonized the chemotaxis mediated by 1 nM CX3CL1 with an apparent affinity of 3-6 nM (FIG. 2D). It also consistently inhibited the calcium response mediated by 20 nM CX3CL1, with an apparent affinity of 34 nM (FIG. 3B). Yet, the apparent F1 affinity in the cell adhesion assay was close to 150 nM, that is, 40 times higher than the apparent efficacy of soluble CX3CL1. This indicates that the effect of F1 on cell avidity for immobilized CX3CL1 did not follow its binding affinity to CX3CR1 and that more complex processes are probably involved in CX3CL1/CX3CR1 adhesion, compared with simple CX3CL1/CX3CR1 binding.

In mice, two major monocyte populations have been described according to the expression levels of CX3CR1 and Ly6C or 7/4. The so-called classical or inflammatory monocytes, which correspond to human CD14⁺ monocytes, are CX3CR1^{lo}Ly6C^{hi}7/4^{hi}CCR2⁺CD62L⁺, whereas the nonclassical monocytes, similar to human CD16⁺ monocytes, are CX3CR1^{hi}Ly6C^{lo}7/4^{lo}CCR2⁻CD62L⁻. Classical monocytes are reported to be recruited rapidly to inflammation sites, independently of CX3CR1 expression. Less is known about nonclassical monocytes, but it has been suggested that they use CX3CR1 to migrate into noninflamed tissue, where they replace resident macrophages or DCs. Recent reports indicate that this monocyte subpopulation patrols the luminal surface of vessels and rapidly infiltrates the tissues to differentiate into macrophages, while classical monocytes reach the inflammatory site later and give rise to inflammatory dendritic cells. The fact that the CX3CR1 antagonist F1 specifically decreases the migration of the CX3CR1-positive monocytic population indicates that antagonists such as F1 might be useful in a thorough analysis of the inflammation pathway and, in a therapeutic setting, in the ultimate prevention of the side-effects of broad-spectrum inhibitors.

Selective CX3CR1 inhibitors such as F1 will prove valuable in controlling inflammation in the various diseases in which CX3CL1 plays a role. Nevertheless, it is very important to direct the action of any CX3CR1 antagonist to the inflamed organ. In diseases such as e.g. atherogenesis or glomerulonephritis, the antagonist will primarily target the circulating and infiltrating monocytes and the resident cells will be inaccessible. In other diseases, specific targeting might be obtained by using a bivalent Ig-chimera. Hence, one antibody specific for the cell marker could be fused to the modulator in accordance with the invention.

### Example 8

### Characterization of F2 as a CX3CR1 Agonist

A polypeptide of SEQ ID NO: 66 (referred to as F2) and a polypeptide of SEQ ID NO: 68 were produced by chemical synthesis. These two polypeptides comprise the sequence of SEQ ID NO: 51 at their N-terminal extremity. They are thus agonists according to the invention. Experimental studies were carried out in order to confirm that these polypeptide act as agonists of the CX3CR1 receptor.

In all experiments, F2 was compared with CX3CL1, and F2-Ig was compared with CX3CL1-Ig. These molecules correspond to proteins similar to F2 and to F2-Ig respectively, but comprising a wild-type N-terminal extremity.

Assays for determining the competitive radioligand binding of F2 and of F2-Ig to CX3CR1 were carried out as described in Example 1.8. As shown on FIG. 4, it was found that F2 and F2-Ig exhibit a significantly higher affinity for CX3CR1 than FKN (CX3CL1) and FKN-Ig (CX3CL1-Ig) respectively. On CX3CR1-expressing HEK cells and with ¹²⁵I-FKN as a tracer, an IC₅₀ of only 0.05 nM was observed for F2 (vs. an IC50 of 0.58 for CX3CL1). On CHO-expressing HEK cells and with ¹²⁵I-FKN as a tracer, an IC50 of only 0.39 nM was observed for F2-Ig (vs. an IC50 of 1.47 for CX3CL1-Ig). In summary, F2 and F2-ig exhibit an affinity for CX3CR1 that is about ten times higher than the affinity of CX3CL1 for the same receptor. This result was found in two different cell types (HEK and CHO).

F2 and F2-Ig were further characterized by carrying out:
an adhesion assay as described in Example 1.11.;
a down-modulation assay as described in Example 1.7.; and
a recycling assay as described in Example 1.8.

It was found that F2-Ig presents a better adherence than CX3CL1-Ig (FIG. 5A). In addition, F2 is capable of inducing the internalization of the membrane CX3CR1 receptor as efficiently as CX3CL1 (FIG. 5B). Moreover, F2 retains the internalized CX3CR1 more efficiently than CX3CL1. Indeed, cells incubated in the presence of F2 recycle the internalized CX3CR1 with a speed that is about two fold slower than the speed observed in the presence of CX3CL1 (FIG. 5C).

The capacity of F2 and F2-Ig of inducing chemotaxis of NK cells, of CD8+ T cells and of CD4+ T cells was further assessed as described in Example 1.10. It was found that F2 and F2-Ig are capable of inducing chemotaxis in all these cells (FIG. 6). This capacity is at least as high as that of CX3CL1-Ig.

The capacity of F2-Ig of inducing a calcium response in CHO-CX3CR1 cells was assessed as described in Example 1.9. It was found that F2-Ig is capable of inducing a calcium response in CHO-CX3CR1 cells (FIG. 7). This capacity is at least as high as that of CX3CL1-Ig.

In summary, the above results demonstrate that F2 and F2-Ig act as agonists of the CX3CR1 receptor. Indeed, F2 and F2-Ig are both capable of (i) inducing chemotaxis of NK cells, of CD8+ T cells and of CD4+ T cells, and (ii) inducing a calcium response. In addition, they exhibit a better affinity to the CX3CR1 receptor than the wild-type CX3CL1 chemokine, as well as a better adherence and a better retention capacity.

It has thus been shown that the functional screening for CX3CR1 agonists described herein allowed the successful isolation of CX3CR1 agonists.

1. An isolated and/or purified modulator of a human CX3CR1 receptor comprising a sequence X₁-X₂-X₃-X₄-X₅-X₆-X₇ (SEQ ID NO: 1) or X₁-X₃-X₄-X₅-X₆-X₇ (SEQ ID NO:2) at its N-terminal extremity, wherein:
X₁ is I, T, F, Q, S, W, A, G, N or V;
X₂, when present, is L, P or R;
X₃ is D, A, Q, G, L, I, P, H, F, V or S;
X₄ is N, Q, G, S, L, R, F, H, V, M, Y or P;
X₅ is V, A, D or G;
X₆ is L, M or V; and
X₇ is S, P, T or A;
and wherein SEQ ID NO: 2 does not consist of the sequence QHHGVT (SEQ ID NO: 52) or QHLGMT (SEQ ID NO: 53).

2. The modulator of claim 1, wherein said modulator is an antagonist, and wherein said sequence of SEQ ID NO: 1 or 2 is selected from the group consisting of:
(i) a sequence X₁-X₂-X₃-X₄-X₅-X₆-X₇ (SEQ ID NO: 1) or X₁-X₃-X₄-X₅-X₆-X₇ (SEQ ID NO: 2) wherein:
X₁ is I, T, F, Q, S, W or V;
X₂, when present, is L, P or R;
X₃ is D, A, Q, G, L, I, P or S;
X₄ is N, Q, G, S, L, R, F, H or P;
X₅ is V, A, D or G;
X₆ is L or V; and
X₇ is 5, P, T or A;
(ii) a sequence X₁-X₂-X₃-X₄-X₅-X₆-X₇ (SEQ ID NO: 1) wherein:
X₁ is I, T, F, Q, S or W;
X₂ is L, P or R;
X₃ is D, A, Q, G, L, I, P or S;
X₄ is N, Q, G, S, L, R, F, H or P;
X₅ is V, A, D or G;
X₆ is L or V; and
X₇ is S, P, T or A;
(iii) a sequence X₁-X₃-X₄-X₅-L-X₇ (SEQ ID NO: 3) wherein:
X₁ is Q or V;
X₃ is Q, L or S;
X₄ is S, L, or F;
X₅ is V or A; and
X₇ is S or P;
(iv) a sequence X₁-L-X₃-X₄-X₅-X₆-X₇ (SEQ ID NO: 4) wherein:
X₁ is I, T, F, S or W;
X₃ is D, A, Q, G, I, P or S;
X₄ is N, Q, G, S, L, R, H or P;
X₅ is V, D or G;
X₆ is L or V; and
X₇ is S, P, T or A;
(v) a sequence Q-X₂-X₃-X₄-X₅-X₆-A (SEQ ID NO: 5) wherein:
X₂ is P or R;
X₃ is D or L;
X₄ is S or F;
X₅ is V or A; and
X₆ is L or V;
(vi) a sequence I-L-D-X₄-G-X₆-X₇ (SEQ ID NO: 6) wherein:
X₄ is any amino acid;
X₆ is L or V; and
X₇ is A or S.

3. The modulator of claim 2, wherein said sequence of SEQ ID NO: 1 or 2 is selected from the group consisting of:

| | |
|---|---|
| ILDNGVS; | (SEQ ID NO: 8) |
| TLAQGLP; | (SEQ ID NO: 9) |
| ILDGGVS; | (SEQ ID NO: 10) |
| FLQSDVA; | (SEQ ID NO: 11) |
| ILDLGLS; | (SEQ ID NO: 12) |
| ILDLGLT; | (SEQ ID NO: 13) |
| ILDNGVA; | (SEQ ID NO: 14) |
| ILGRDVA; | (SEQ ID NO: 15) |
| QPLFAVA; | (SEQ ID NO: 16) |
| QRDSVLA; | (SEQ ID NO: 17) |
| SLDHGLS; | (SEQ ID NO: 18) |
| SLIPWP; | (SEQ ID NO: 19) |
| TLPQGLA; | (SEQ ID NO: 20) |
| WLSQGLA; | (SEQ ID NO: 21) |
| QSLVLP; | (SEQ ID NO: 22) |
| QLFALS; | (SEQ ID NO: 23) |
| and | |
| VQSVLS. | (SEQ ID NO: 24) |

4. The modulator of claim 1, wherein said modulator is an agonist, and wherein said sequence of SEQ ID NO: 1 or 2 is a sequence X₁-X₃-X₄-X₅-X₆-X₇ (SEQ ID NO:2) in which:
X₁ is Q, A, G or N;
X₃ is P, A, H, L, S, F or V;
X₄ is G, Q, V, M, L, S, P, H, R or Y;
X₅ is A or G;
X₆ is L, M or V; and
X₇ is 5, P, T or A.

5. The modulator of claim 4, wherein said sequence of SEQ ID NO: 2 is selected from the group consisting of:

| | |
|---|---|
| QPGGVS; | (SEQ ID NO: 25) |
| QPQAVS; | (SEQ ID NO: 26) |
| QPVALA; | (SEQ ID NO: 27) |
| QPVGLS; | (SEQ ID NO: 28) |
| AAQGMS; | (SEQ ID NO: 29) |
| QPGAVS; | (SEQ ID NO: 30) |
| QPMGVA; | (SEQ ID NO: 31) |
| QPQGLA; | (SEQ ID NO: 32) |
| QPVAVA; | (SEQ ID NO: 33) |
| QHLGLS; | (SEQ ID NO: 34) |
| QLQGLA; | (SEQ ID NO: 35) |
| QPSALS; | (SEQ ID NO: 36) |
| QSLGVS; | (SEQ ID NO: 37) |
| GPQAMS; | (SEQ ID NO: 38) |
| NPQALS; | (SEQ ID NO: 39) |
| QFPGVS; | (SEQ ID NO: 40) |
| QLLGVS; | (SEQ ID NO: 41) |
| QPHGVA; | (SEQ ID NO: 42) |
| QPRALP; | (SEQ ID NO: 43) |
| QPSALT; | (SEQ ID NO: 44) |
| QPSGMS; | (SEQ ID NO: 45) |
| QPVAVS; | (SEQ ID NO: 46) |
| QPYGMS; | (SEQ ID NO: 47) |
| QPYGVS; | (SEQ ID NO: 48) |
| QSPGMS; | (SEQ ID NO: 49) |
| QVQGVT; | (SEQ ID NO: 50) |
| and | |
| QPQGVS. | (SEQ ID NO: 51) |

6. An isolated and/or purified modulator of a human CX3CR1 receptor, wherein said modulator is an agonist comprising a sequence X₁-X₃-X₄-X₅-X₆-X₇ (SEQ ID NO: 7) at its N-terminal extremity, and wherein:
X₁ is any amino acid;
X₃ is any amino acid;
X₄ is any amino acid;
X₅ is V, A, D or G;
X₆ is L, M or V; and
X₇ is S, P, T or A;
and wherein SEQ ID NO: 7 does not consist of the sequence QHHGVT (SEQ ID NO: 52) or QHLGMT (SEQ ID NO: 53).

7. The modulator of claim 1, wherein said modulator consists of a polypeptide comprising a fragment of at least ten amino acids of SEQ ID NO: 55 or 56.

8. The modulator of claim 7, wherein said polypeptide comprises or consists of amino acids 1 to 77 of SEQ ID NO: 55 or of amino acids 1 to 76 of SEQ ID NO: 56.

9. The modulator of claim 7, wherein the N-terminal extremity of said modulator consists of the sequence of any one of SEQ ID NOs. 8 to 51.

10. The modulator of claim 7, wherein said polypeptide further comprises a fragment of an immunoglobulin.

11. The modulator of claim 1, wherein said modulator is a peptide.

12. An isolated and/or purified mutant of a human CX3CL1 polypeptide characterized in that the N-terminal extremity of a mature isoform of said mutant:
consists of the sequence of any one of SEQ ID NOs. 1 to 51; and
does not consist of QHHGVT (SEQ ID NO: 52) or QHLGMT (SEQ ID NO: 53).

13. A nucleic acid encoding a modulator of a human CX3CR1 receptor or a mutant of a human CX3CL1 polypeptide wherein:
said receptor comprises a sequence X₁-X₂-X₃-X₄-X₅-X₆-X₇ (SEQ ID NO: 1) or X₁-X₃-X₄-X₅-X₆-X₇ (SEQ ID NO:2) at its N-terminal extremity, in which:
X₁ is I, T, F, Q, S, W, A, G, N or V;
X₂, when present, is L, P or R;
X₃ is D, A, Q, G, L, I, P, H, F, V or S;
X₄ is N, Q, G, S, L, R, F, H, V, M, Y or P;
X₅ is V, A, D or G;
X₆ is L, M or V; and
X₇ is S, P, T or A;
and in which SEQ ID NO: 2 does not consist of the sequence QHHGVT (SEQ ID NO: 52) or QHLGMT (SEQ ID NO: 53); and
wherein said mutant is characterized in that the N-terminal extremity of a mature isoform of said mutant:
consists of the sequence of any one of SEQ ID NOs. 1 to 51; and
does not consist of QHHGVT (SEQ ID NO: 52) or QHLGMT (SEQ ID NO: 53).

14. A pharmaceutical composition comprising a modulator according to claim 1, a mutant according to claim 12, or a nucleic acid according to claim 13, and a physiologically acceptable carrier.

15. The pharmaceutical composition of claim 14, wherein said pharmaceutical composition is a vaccine comprising an immunogenic molecule.

16. The pharmaceutical composition of claim 14, wherein said physiologically acceptable carrier is a hydrogel matrix, and wherein said modulator, polypeptide or nucleic acid is covalently bound into the hydrogel matrix.

17. A method for treating or preventing a disease selected from the group consisting of an inflammatory disorder, an autoimmune disorder, a cardiovascular disease, a neurodegenerative disease, a graft versus host disease, a behavioral disorder, a cicatrisation disorder, a viral infection, cancer and pain comprising the step of administering an effective amount of modulator according to claim 1, a mutant according to claim 12, or a nucleic acid according to claim 13, to an individual in need thereof.

18. The method of claim 17, wherein said modulator is administered through a parenteral or topical route.

19. The method of claim 17, wherein said modulator is an antagonist and said disease is selected from the group consisting of an inflammatory disorder, an autoimmune disorder, a cardiovascular disease, a neurodegenerative disease, cancer and a graft versus host disease.

20. The method of claim 19, wherein said disease is an autoimmune disorder selected from the group consisting of multiple sclerosis, rheumatoid arthritis, lupus erythematosus, inflammatory bowel disease and ulcerative colitis.

21. The method of claim 19, wherein said disease is atherosclerosis.

22. The method of claim 19, wherein said disease is a cancer selected from breast cancer, colon cancer and lymphoma.

23. The method of claim 17, wherein said modulator is an agonist and said disease is selected from the group consisting of a viral infection and a behavioral disorder such as a disturbance of activity and attention.

24. The method of claim 23, wherein said disease is an HIV infection.

25. A method for stimulating an anti-tumoral response or cicatrisation comprising the step of administering an effective amount of modulator according to claim 1, a mutant according to claim 12, or a nucleic acid according to claim 13, to an individual in need thereof.

26. A method for vaccinating an individual comprising the step of administering a vaccine composition comprising an effective amount of an agonist according to claim 1, a mutant according claim 12, or a nucleic acid according to claim 13, to said individual.

27. A method of producing the modulator according to claim 1 or the mutant according to claim 12 comprising the step of: a) providing a host cell comprising the nucleic acid according to claim 13; b) cultivating said host cell under conditions suitable for the expression of said modulator or mutant; and c) isolating said modulator or mutant.

28. The method of claim 27, further comprising the step of purifying said modulator or mutant.

29. The method of claim 28, further comprising the step of formulating said modulator or mutant into a pharmaceutical composition.


SEQUENCE LISTING (70 sequences)

SEQ ID NO: 1 - protein, 7 aa, Artificial
  CX3CR1 modulator
XXXXXXX

SEQ ID NO: 2 - protein, 6 aa, Artificial
  CX3CR1 modulator
XXXXXX

SEQ ID NO: 3 - protein, 6 aa, Artificial
  CX3CR1 modulator
XXXXLX

SEQ ID NO: 4 - protein, 7 aa, Artificial
  CX3CR1 modulator
XLXXXXX

SEQ ID NO: 5 - protein, 7 aa, Artificial
  CX3CR1 modulator
QXXXXXA

SEQ ID NO: 6 - protein, 7 aa, Artificial
  CX3CR1 modulator
ILDXGXX

SEQ ID NO: 7 - protein, 6 aa, Artificial
  CX3CR1 modulator
XXXXXX

SEQ ID NO: 8 - protein, 7 aa, Artificial
  CX3CR1 modulator
ILDNGVS

SEQ ID NO: 9 - protein, 7 aa, Artificial
  CX3CR1 modulator
TLAQGLP

SEQ ID NO: 10 - protein, 7 aa, Artificial
  CX3CR1 modulator
ILDGGVS

SEQ ID NO: 11 - protein, 7 aa, Artificial
  CX3CR1 modulator
FLQSDVA

SEQ ID NO: 12 - protein, 7 aa, Artificial
  CX3CR1 modulator
ILDLGLS

SEQ ID NO: 13 - protein, 7 aa, Artificial
  CX3CR1 modulator
ILDLGLT

SEQ ID NO: 14 - protein, 7 aa, Artificial
  CX3CR1 modulator
ILDNGVA

SEQ ID NO: 15 - protein, 7 aa, Artificial
  CX3CR1 modulator
ILGRDVA

SEQ ID NO: 16 - protein, 7 aa, Artificial
  CX3CR1 modulator
QPLFAVA

SEQ ID NO: 17 - protein, 7 aa, Artificial
  CX3CR1 modulator
QRDSVLA

SEQ ID NO: 18 - protein, 7 aa, Artificial
  CX3CR1 modulator
SLDHGLS

SEQ ID NO: 19 - protein, 7 aa, Artificial
  CX3CR1 modulator
SLIPVVP

SEQ ID NO: 20 - protein, 7 aa, Artificial
  CX3CR1 modulator
TLPQGLA

SEQ ID NO: 21 - protein, 7 aa, Artificial
  CX3CR1 modulator
WLSQGLA

SEQ ID NO: 22 - protein, 6 aa, Artificial
  CX3CR1 modulator
QSLVLP

SEQ ID NO: 23 - protein, 6 aa, Artificial
  CX3CR1 modulator
QLFALS

SEQ ID NO: 24 - protein, 6 aa, Artificial
  CX3CR1 modulator
VQSVLS

SEQ ID NO: 25 - protein, 6 aa, Artificial
  CX3CR1 modulator
QPGGVS

SEQ ID NO: 26 - protein, 6 aa, Artificial
  CX3CR1 modulator
QPQAVS

SEQ ID NO: 27 - protein, 6 aa, Artificial
  CX3CR1 modulator
QPVALA

SEQ ID NO: 28 - protein, 6 aa, Artificial
  CX3CR1 modulator
QPVGLS

SEQ ID NO: 29 - protein, 6 aa, Artificial
  CX3CR1 modulator
AAQGMS

SEQ ID NO: 30 - protein, 6 aa, Artificial
  CX3CR1 modulator
QPGAVS

SEQ ID NO: 31 - protein, 6 aa, Artificial
  CX3CR1 modulator
QPMGVA

SEQ ID NO: 32 - protein, 6 aa, Artificial
  CX3CR1 modulator
QPQGLA

SEQ ID NO: 33 - protein, 6 aa, Artificial
  CX3CR1 modulator
QPVAVA

SEQ ID NO: 34 - protein, 6 aa, Artificial
  CX3CR1 modulator
QHLGLS

SEQ ID NO: 35 - protein, 6 aa, Artificial
  CX3CR1 modulator
QLQGLA

SEQ ID NO: 36 - protein, 6 aa, Artificial
  CX3CR1 modulator
QPSALS

SEQ ID NO: 37 - protein, 6 aa, Artificial
  CX3CR1 modulator
QSLGVS

SEQ ID NO: 38 - protein, 6 aa, Artificial
  CX3CR1 modulator
GPQAMS

SEQ ID NO: 39 - protein, 6 aa, Artificial
  CX3CR1 modulator
NPQALS

SEQ ID NO: 40 - protein, 6 aa, Artificial
  CX3CR1 modulator
QFPGVS

SEQ ID NO: 41 - protein, 6 aa, Artificial
  CX3CR1 modulator
QLLGVS

SEQ ID NO: 42 - protein, 6 aa, Artificial
  CX3CR1 modulator
QPHGVA

SEQ ID NO: 43 - protein, 6 aa, Artificial
  CX3CR1 modulator
QPRALP

SEQ ID NO: 44 - protein, 6 aa, Artificial
  CX3CR1 modulator
QPSALT

SEQ ID NO: 45 - protein, 6 aa, Artificial
  CX3CR1 modulator
QPSGMS

SEQ ID NO: 46 - protein, 6 aa, Artificial
  CX3CR1 modulator
QPVAVS

SEQ ID NO: 47 - protein, 6 aa, Artificial
  CX3CR1 modulator
QPYGMS

SEQ ID NO: 48 - protein, 6 aa, Artificial
  CX3CR1 modulator
QPYGVS

SEQ ID NO: 49 - protein, 6 aa, Artificial
  CX3CR1 modulator
QSPGMS

SEQ ID NO: 50 - protein, 6 aa, Artificial
  CX3CR1 modulator
QVQGVT

SEQ ID NO: 51 - protein, 6 aa, Artificial
  CX3CR1 modulator
QPQGVS

SEQ ID NO: 52 - protein, 6 aa, Homo sapiens
  MISC_FEATURE (1)..(6): N-terminal extremity of native human CX3CL1
QHHGVT

SEQ ID NO: 53 - protein, 6 aa, Mus musculus
  MISC_FEATURE: N-terminal extremity of native murine CX3CL1
QHLGMT

SEQ ID NO: 54 - protein, 397 aa, Homo sapiens
  SIGNAL (1)..(24)
  CHAIN (25)..(397): Fraktaline
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 55 - protein, 374 aa, Artificial
  mutant CX3CL1
XXXXXXXKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 56 - protein, 373 aa, Artificial
  mutant CX3CL1
XXXXXXKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 57 - protein, 336 aa, Artificial
  chimeric polypeptide
MAPISLSWLLRLATFCHLTVLLAGILDNGVSCNITCSKMTSKIPVALLIHYQQNQASCGKRAIILETRQHRLFCADPKEQWVKDAMQHLDRQAAALTRNGAAAHPRGPTIKPCPPCKCPAPNLEGGPSVFIFPPKIKDVLMISLSPIVTCVVVDVSEDDPDVQISWFVNNVEVHTAQTQTHREDYDSTLRVVSALPIQHQDWMSGKAFACAVNNKDLPAPIGRTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRPPGK

SEQ ID NO: 58 - DNA, 1194 bp, Homo sapiens
atggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcccggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtttaa

SEQ ID NO: 59 - DNA, 29 bp, Artificial
  oligonucleotide
ccggccatggcccagcaccacggtgtgac

SEQ ID NO: 60 - DNA, 30 bp, Artificial
  oligonucleotide
ttgttctgcggccgcgccatttcgagttag

SEQ ID NO: 61 - DNA, 51 bp, Artificial
  oligonucleotide
ccggccatggccnnkcnannknnkgncntgncaaaatgcaacatcacgtgc

SEQ ID NO: 62 - DNA, 105 bp, Artificial
  oligonucleotide
aaaactgcagccatggctccgatatctctgtcgtggctgctccgcttggccaccttctgccatctgactgtcctgctggctggaattctagataatggcgtgtca

SEQ ID NO: 63 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 64 - protein, 7 aa, Artificial
  CX3CR1 modulator
XXXXXXX

SEQ ID NO: 65 - DNA, 228 bp, Artificial
  F2 agonist according to the invention (F2)
cagcctcagggcgtgtcaaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggc

SEQ ID NO: 66 - protein, 76 aa, Artificial
  Synthetic Construct
QPQGVSKCNITCSKMTSKIPVALLIHYQQNQASCGKRAIILETRQHRLFCADPKEQWVKDAMQHLDRQAAALTRNG

SEQ ID NO: 67 - DNA, 936 bp, Artificial
  F2-Ig agonist according to the invention
cagcctcagggcgtgtcaaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcgcggccgcacatcccagagggcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcaaacacaaacccatagagaggattacgacagtactctccgggtggtcagtgccctccccatccagcaccaggactggatgagtggcaaggcattcgcatgcgcagtcaacaacaaagaccttccagcgcccatcgggagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggcctccgggtaaa

SEQ ID NO: 68 - protein, 312 aa, Artificial
  Synthetic Construct
QPQGVSKCNITCSKMTSKIPVALLIHYQQNQASCGKRAIILETRQHRLFCADPKEQWVKDAMQHLDRQAAALTRNGAAAHPRGPTIKPCPPCKCPAPNLEGGPSVFIFPPKIKDVLMISLSPIVTCVVVDVSEDDPDVQISWFVNNVEVHTAQTQTHREDYDSTLRVVSALPIQHQDWMSGKAFACAVNNKDLPAPIGRTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRPPGK

SEQ ID NO: 69 - DNA, 48 bp, Artificial
  primer
ccggccatggccnnknnknnkgncntgncaaaatgcaacatcacgtgc

SEQ ID NO: 70 - DNA, 105 bp, Artificial
  primer
aaaactgcagccatggctccgatatctctgtcgtggctgctccgcttggccaccttctgccatctgactgtcctgctggctggacagcctcagggcgtgtcaaaa